(19) United States
(12) Patent Application Publication
(10) Pub. No.: US 2003/0207886 A1
(43) Pub. Date: Nov. 6, 2003

(54) Preparation containing quinoxaline derivatives

(76) Inventors: Frank Plücker, (US); Hansj?uuml;rgen Driller, Gross-Umstadt (DE); Michael Kirschbaum, Weiterstadt (DE); Volker Scholz, Darmstadt (DE); Hans Neunhoeffer, M?uuml;hltal (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/221,726

(22) PCT Filed: Mar. 6, 2001
(86) PCT No.: PCT/EP01/02517

(30) Foreign Application Priority Data
Mar. 17, 2000 (DE) 100 13 318.5

(51) Int. Cl. A61K 31/498

(52) U.S. Cl. 514/249

(57) ABSTRACT

The invention relates to the use of quinoxaline derivatives as photostable UV filters in cosmetic and pharmaceutical preparations for protecting the human epidermis or human hair against UV radiation, especially in the 280-400 nm range.

The invention relates to the use of quinoxaline derivatives as photostable UV filters in cosmetic and pharmaceutical preparations for protection of the human epidermis or human hair against UV radiation, especially in the range 280-400 nm.

The light-protection agents employed in cosmetic and pharmaceutical preparations have the job of preventing or at least reducing the effects of harmful influences of sunlight on the human skin. In addition, however, these light-protection agents also serve to protect further ingredients against destruction or degradation by UV radiation. In hair-cosmetic preparations, the aim is to prevent damage to the keratin fibres by UV rays.

As is known, the skin reacts sensitively to solar radiation, which can cause normal sunburn or erythema, but also various degrees of burning.

However, solar radiation also has other adverse effects: it causes the skin to lose its elasticity and causes the formation of wrinkles and thus results in premature ageing. Dermatosis is sometimes also observed, and in the extreme case skin cancer can arise.

Owing to this knowledge, changes have also occurred in sun protection. Whereas the main aim a few years ago was erythema-preventing UV-B protection, protection against UV-A radiation is now included in sun-protection preparations.

UV-A radiation is essentially the trigger for pigmentation of the skin.

It is also desirable to protect hair against photochemical damage in order to prevent changes in colour shade, bleaching or damage of a mechanical nature.

As is known, the most dangerous part of solar radiation is formed by ultraviolet rays having a wavelength of less than 400 nm. It is also known that the presence of the ozone layer in the earth's atmosphere, which absorbs part of solar radiation, means that the lower limit for the ultraviolet rays which reach the earth's surface is about 280 nm. All this knowledge therefore makes the development of efficient filter substances for the UV-A and also for the UV-B region appear necessary.

There is a growing demand for light-protection agents for cosmetic and pharmaceutical preparations which can serve, in particular, as UV-A filters and whose absorption maxima should therefore be in the range from about 320 to 400 nm. There is furthermore also a demand for broad-band protection, i.e. UV-A and UV-B protection, in the range 280-400 nm.

In order to achieve the desired effect using the smallest possible amount, light-protection agents of this type should additionally have highly specific absorbance. In addition, light-protection agents for cosmetic preparations have to meet a multiplicity of further demands, for example good solubility in cosmetic oils or in water, high stability of the emulsions prepared therewith, toxicological acceptability and low inherent odour and low inherent colour.

A further requirement that light-protection agents have to satisfy is adequate photostability. However, this is often only guaranteed to an inadequate extent with the UV-A and UV-B absorbing light-protection agents available hitherto.

Although the prior art contains various approaches for improving the photostability of good light-protection filters, such as, for example, of dibenzoylmethanes, by combination with various UV-B filters (FR 2 440 933), or also for stabilising the UV filters by addition of certain substances (EP 0514491), adequate solutions are still, however, not available therewith.

Furthermore, DE-A 197 46 656 and EP 0 852 137 propose substance classes, such as 4,4-diarylbutadienes or compounds containing an R⁴—NH—CR³═CR¹R² structure, as novel light-protection filters, but these do not adequately meet the demand for suitable compounds for the UV-A and UV-B regions.

The object was therefore to find a novel structural class as light-protection agents for cosmetic and pharmaceutical purposes which absorb in the UV-A and/or UV-B region, are photostable, have low inherent colour, i.e. a sharp band structure, have high absorbance and are soluble in oil or water, depending on the substituent.

It has been found that quinoxaline derivatives containing a wide variety of radicals have excellent UV-B and/or UV-A properties and meet the above-described requirements to a high extent. The object has accordingly been achieved in accordance with the invention through the use of quinoxaline derivatives of the formulae I, II and/or III.

Although similar quinoxaline derivatives are described in EP 0 728 481 A2, these already known compounds are used therein as medicaments for the treatment of AIDS and/or HIV infections.

The invention therefore relates to the use of quinoxaline derivatives of the formulae I, II and/or III

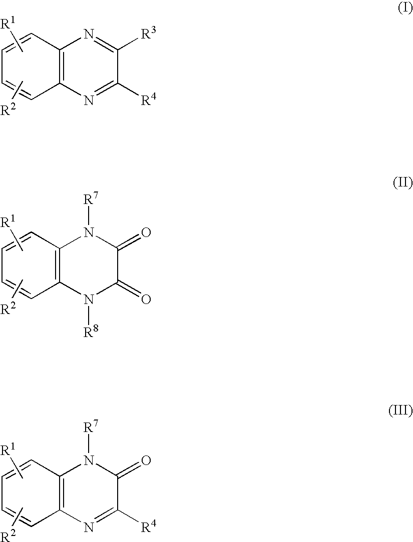

in which

R¹and R² are each, independently of one another, H,

alkyl, alkoxy, alkenyl or alkynyl, each having up to 20 carbon atoms, cycloalkyl, cycloalkoxy, cycloalkenyl or bicyclic systems, each having up to 10 carbon atoms, where, in each of these groups, one or more hydrogen atoms may also be substituted by Sub¹ and/or one or two CH₂ groups may be replaced by C═O, and the cyclic systems may contain from 1 to 3 heteroatoms, such as S, N and/or O,

Hal, OH, NO₂, —(CR⁵R⁶)ₙ—NR⁵R⁶, —(CR⁵R⁶)ₙ—N═CR⁵R⁶, —(CR⁵R⁶)ₙ—CR⁵═NR⁵, —(CR⁵R⁶)ₙ—NHCOR⁵, —(CR⁵R⁶)ₙ—NHCOOR⁵, —SR⁵, —SO₂—R⁵ or NR⁵—SO—R⁶, —SO—R⁵, water-solubilising substituents selected from the group consisting of carboxylate, sulfonate or ammonium radicals, COR⁵, COOR⁵, COR⁵R⁶, CN, O═S(—R⁵)═O, O═S(—OR⁵)═O, O═S(—NR⁵R⁶)═O, R⁵OP(—OR⁶)═O, OAr, —(CR⁵R⁶)ₙ—Ar, -Het, —NHHet, —OHet or —(CR⁵R⁶)ₙ-Het,

R¹ and R² together, also with carbon atoms to which they are bonded, may jointly form an unsaturated, partially saturated or fully saturated 4-, 5-, 6- or 7-membered ring, which may optionally contain heteroatoms, such as S, N and/or O, may be further fused and/or may also be monosubstituted or polysubstituted,

Sub¹ is Hal, hydroxyl, cyano, amino, nitro, C₁-C₄-alkylamino, C₁-C₄-dialkylamino, C₁-C₄-alkyl or C₁-C₄-alkoxy, COOH or COO-alkyl,

Hal is fluorine, chlorine, bromine or iodine,

n is 0, 1, 2, 3 or 4,

R⁵ and R⁶ are each, independently of one another, H,

alkyl, alkenyl or alkynyl, each having up to 20 carbon atoms, cycloalkyl, cycloalkenyl or bicyclic systems, each having up to 10 carbon atoms, where these radicals may be up to trisubstituted by Sub¹, and/or one or two CH₂ groups may be replaced by C═O, and the cyclic systems may also contain from 1 to 3 heteroatoms, such as S, N and/or O, —(CR^{′}R^{″})ₙ—Ar or —(CR^{′}R^{″})ₙ-Het,

the radicals R⁵ and R⁶ may also jointly form with one another, in each case together with carbon atoms to which they are bonded, an unsaturated, partially saturated or fully saturated 4-, 5-, 6- or 7-membered ring, which may optionally contain heteroatoms, such as S, N and/or O, may also be mono-substituted or polysubstituted and/or may be further fused,

R^{′} and R^{″} are each, independently of one another, H or C₁-C₄-alkyl, where one or two CH₂ groups may also be replaced by C═O,

Ar is an unsubstituted or monosubstituted or polysubstituted aromatic ring or fused ring system having from 6 to 18 carbon atoms, in which, in addition, one or two CH groups in the α- or β-position to the heteroatoms may be replaced by C═O,

Het is an unsubstituted or monosubstituted or polysubstituted heteroaromatic ring having from 5 to 7 ring members or a fused ring system, in which one or more N, S and/or O atoms are present as heteroatoms and in which, in addition, one or two CH groups in the α- or β-position to the heteroatoms may be replaced by C═O,

R³ and R⁴ are each, independently of one another, H,

alkyl, alkoxy, alkenyl or alkynyl, each having up to 20 carbon atoms, cycloalkyl, cycloalkoxy, cycloalkenyl or bicyclic systems, each having up to 10 carbon atoms, where these radicals may be up to trisubstituted by Sub² and/or one or two CH₂ groups may be replaced by C═O and where the cyclic systems may also contain from 1 to 3 heteroatoms, such as S, N and/or O,

Hal, OH, NO₂, —(CR⁵R⁶)ₙ—NR⁵R⁶, —(CR⁵R⁶)ₙ—N═CR⁵R⁶, —(CR⁵R⁶)ₙ—CR⁵═NR⁵, —(CR⁵R⁶)ₙ—NHCOR⁵, —(CR⁵R⁶)ₙ—NHCOOR⁵, —SR⁵, —SO₂—R⁵, NR⁵—SO—R⁶ or —SO—R⁵, water-solubilising substituents selected from the group consisting of carboxylate, sulfonate or ammonium radicals, COR⁵, COOR⁵, CONR⁵R⁶, CN, O═S(—R⁵)═O, O═S(—OR⁵)═O, O═S(—NR⁵R⁶)═O, R⁷OP(—OR⁸)═O, OAr, —(CR⁵R⁶)ₙ—Ar, -Het, —NHHet, —OHet or —(CR⁵R⁶)-Het,

the radicals R³ and R⁴ may also jointly form with one another, in each case together with the carbon atoms to which they are bonded, or alternatively one of the two radicals together with the adjacent N atom, an unsaturated, partially saturated or fully saturated 4-, 5-, 6- or 7-membered ring, which may optionally contain heteroatoms, such as S, N and/or O, may also be monosubstituted or polysubstituted and/or may be further fused,

R⁷ and R⁸ are each, independently of one another, H,

alkyl, alkoxy, alkenyl or alkynyl, each having up to 20 carbon atoms, cycloalkyl, cycloalkoxy, cycloalkenyl or bicyclic systems, each having up to 10 carbon atoms, where these radicals may be up to trisubstituted by Sub² and/or one or two CH₂ groups may be replaced by C═O and where the cyclic systems may also contain from 1 to 3 heteroatoms, such as S, N and/or O,

Sub² is Hal, hydroxyl, cyano, amino, nitro, C₁-C₄-alkyl or C₁-C₄-alkoxy, COR⁵, COOR⁵, OAr, OHet, —(CR⁵R⁶)ₙ—Ar or —(CR⁵R⁶)-Het, —(CR⁵R⁶)ₙ—NR⁵R⁶, CONR⁵R⁶, CN, O═S(—R⁵)═O, O═S(—OR⁵)═O, O═S(—NR⁵R⁶)═O or R⁷OP(—OR⁸)═O,

as photostable UV filters in cosmetic and pharmaceutical preparations for protection of the human skin or human hair against solar radiation, alone or together with UV-absorbent compounds known per se for cosmetic and pharmaceutical preparations.

The compounds of the formulae I-III can be prepared substantially analogously to the literature. Preparation processes are described, for example, in

Schipper, Day, I. An. Chem. Soc. 73(1951) 5672 and

Miyashita, Suzuki, Iwamoto, Oishi, Higashino, Heterocycles 49 (1998) 405-413.

For the preparation of illustrative compounds, reference is also made, in particular, to the example part of this application (Examples A-O).

The compounds from this quinoxaline class of substances exhibit excellent UV-absorbent properties both in the UV-A region and, in the presence of an additional chromophoric group, in the UV-B region, thus providing broad-band protection. The solubility of the substances in water or in cosmetic oils can likewise easily be induced through the choice of suitable substituents. Lipophilic radical, i.e. radicals which increase the oil solubility of the compounds of the formulae I, II and/or III, are, for example, aliphatic or cycloaliphatic radicals, in particular alkyl radicals having up to 20 carbon atoms, alkoxy, mono- and dialkylamino, alkoxycarbonyl, mono- and dialkylaminocarbonyl, mono- and dialkylaminosulfonyl radicals, furthermore also cyano, nitro, bromine, chlorine, iodine or fluorine substituents.

Hydrophilic radicals, i.e. radicals which facilitate the water solubility of the compounds of the formulae I, II and/or III, are, for example, carboxyl and sulfoxy radicals and in particular salts thereof with any desired physiologically tolerated cations, such as the alkali metal salts or the trialkylammonium salts.

The alkyl radicals in the radicals R¹ to R⁸ have up to 20 carbon atoms and may be in unbranched or branched form and accordingly are preferably methyl, ethyl, n-propyl, i-propyl, butyl, sec-butyl, i-butyl, tert-butyl, pentyl, 1-methylbutyl, 2-methylbutyl, 3-methylbutyl, hexyl, 1-methylpentyl, 2-methylpentyl, heptyl, octyl, nonyl, decyl, undecyl or dodecyl, furthermore also 2,2-dimethylpropyl, 1-ethylpropyl, 3-methylpentyl, 4-methylpentyl, 1,1-dimethylpropyl, 1,2-dimethylpropyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, tridecyl, tetradecyl, pentadecyl, hexadecyl, heptadecyl, octadecyl, nonadecyl or eicosyl.

Preferred alkenyl radicals which may be mentioned are branched and unbranched alkenyl chains, preferably having up to 10 carbon atoms: vinyl, propenyl, isopropenyl, 1-butenyl, 2-butenyl, 1-pentenyl, 2-pentenyl, 2-methyl-1-butenyl, 2-methyl-2-butenyl, 3-methyl-1-butenyl, 1-hexenyl, 2-hexenyl, 1-heptenyl, 2-heptenyl, octenyl, nonenyl or decenyl.

Suitable alkynyl radicals are preferably branched or unbranched alkynyl chains having up to 10 carbon atoms, such as, for example, ethynyl, propynyl, butynyl, i-butynyl, pentynyl, hexynyl, heptynyl or octynyl.

Cycloalkyl radicals which may be mentioned are preferably branched or unbranched C₃-C₁₀-cycloalkyl chains, such as cyclopropyl, cyclobutyl, cyclopentyl, cyclohexyl, cycloheptyl, cyclooctyl, 1-methylcyclopropyl, 1,2-dimethylcyclopentyl, 1-methyl-2-ethylcyclopropyl, cyclononyl or cyclodecyl.

Suitable alkoxy radicals are branched or unbranched alkoxy chains having up to 20 carbon atoms, preferably having up to 12 carbon atoms, particularly preferably having from 1 to 8 carbon atoms, such as, for example, methoxy, ethoxy, propoxy, i-propoxy, butoxy, 1-methylpropoxy, 2-methylpropoxy, pentoxy, 1,1-dimethylpropoxy, 1-methylbutoxy, 3-methylbutoxy, 2-methylbutoxy, hexoxy, heptoxy or octoxy.

Cycloalkyl radicals which may be mentioned for R¹ to R⁸ are preferably branched or unbranched cycloalkyl chains having 3-10 carbon atoms, such as cyclopropyl, cyclobutyl, cyclopentyl, cyclohexyl, cycloheptyl, 1-methylcyclopropyl, 1-ethylcyclopropyl, 1-propylcyclopropyl, 1,2-dimethylcyclopropyl, 1-methylcyclohexyl, 1,3-dimethylcyclohexyl, cyclooctyl, cyclononyl or cyclodecyl.

Cycloalkenyl radicals which may be mentioned are preferably branched or unbranched C₃-C₁₀-cycloalkenyl chains having one or more double bonds, such as cyclopropenyl, cyclobutenyl, cyclopentenyl, cyclopentadienyl, cyclohexenyl, 1,3-cyclohexadienyl, 1,4-cyclohexadienyl, cycloheptenyl, cycloheptatrienyl, cyclooctenyl, cyclononenyl or cyclodecenyl.

Bicycloalkyl or bicycloalkenyl radicals which may be mentioned are saturated or unsaturated bicyclic ring systems, preferably having up to 10 carbon atoms, preferably bicyclic terpenes, such as pinane, pinene, bornane or camphor derivatives, decalin or adamantane.

These cyclic systems may also contain from 1 to 3 heteroatoms, such as sulfur, nitrogen or oxygen. Examples thereof which may be mentioned are ring systems, such as piperidine, pyrrolidine, pyrazidine, morpholine, tetrahydrofuran, dihydrofuran, thiolane, piperazine, thiazolidine or oxazolidine groups.

In the radicals above and below which may be substituted by Sub¹, Sub¹ is preferably halogen, such as fluorine, chlorine, bromine or iodine, preferably fluorine or chlorine, furthermore preferably C₁-C₄-alkylamino or C₁-C₄-dialkylamino, C₁-C₄-alkyl or C₁-C₄-alkoxy, or alternatively hydroxyl or amino.

In the radicals above and below which may be substituted by Sub², Sub² is preferably halogen, such as fluorine, chlorine, bromine or iodine, preferably fluorine or chlorine, furthermore preferably C₁-C₄-alkylamino or C₁-C₄-dialkylamino, C₁-C₄-alkyl or C₁-C₄-alkoxy, or alternatively hydroxyl or amino, further preference also being given to the meaning COR⁵, —(CR⁵R⁶)ₙ—Ar, —(CR⁵R⁶)ₙ-Het, OAr, OHet, COOR⁵ or R⁵OP(—OR⁶)═O.

Suitable mono- or dialkylamino radicals are preferably methylamino, dimethylamino, ethylamino, methylethylamino, diethylamino, propylamino, methylpropylamino, dipropylamino, ethylpropylamino, butylamino, dibutylamino, methylbutylamino or isopropylamino, furthermore also 1,1-dimethylpropylamino, pentylamino, hexylamino, 1-methyl-1-ethylpropylamino, heptylamino or octylamino.

Preference is also given to compounds in which one of the radicals R¹ and R² or one of the radicals R³ and R⁴ is —(CR⁵R⁶)ₙ—NR⁵R⁶, —(CR⁵R⁶)ₙ—N═CR⁵R⁶ or —(CR⁵R⁶)ₙ—CR⁵═NR⁵. R¹ and R² as well as R³ and R⁴ may—like the radicals R⁵ and R⁶ with one another too—jointly form, together with carbon atoms to which they are bonded, an unsaturated, partially saturated or fully saturated 4-, 5-, 6- or 7-membered ring, which may optionally contain heteroatoms, such as S, N and/or O, may be further fused and/or may also be monosubstituted or polysubstituted. In this respect, the following preferred compounds are indicated by way of example, but in no way have a limiting character:

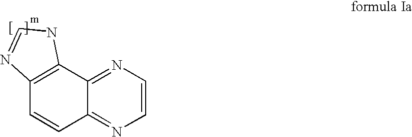

in which m is 1, 2, 3 or 4, preferably 1 or 2;

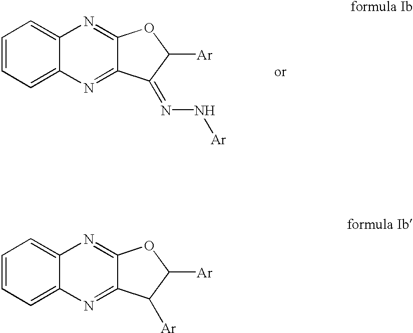

in which Ar is in each case an unsubstituted or monosubstituted or polysubstituted aromatic ring or fused ring systems having from 6 to 18 carbon atoms, in which, in addition, one or two CH groups may be replaced by C═O, preferably phenyl, mono- to trisubstituted phenyl or naphthyl;

or

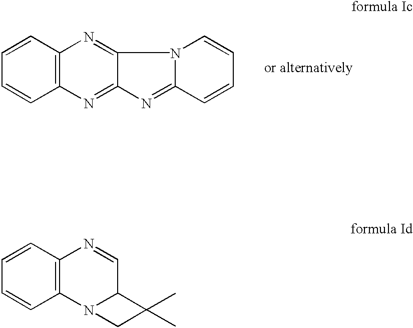

In the compounds and formulae above and below, Ar is an unsubstituted or monosubstituted or polysubstituted aromatic ring or a fused ring system having from 6 to 18 carbon atoms, preferably having from 6 to 10 carbon atoms, in which, in addition, one or two CH groups may be replaced by C═O. Particularly preferred groups which may be mentioned are unsubstituted or substituted phenyl or naphthyl.

Het in the definitions above and below is an unsubstituted or monosubstituted or polysubstituted heteroaromatic ring having from 5 to 7 ring members or a fused ring system, preferably having up to 14 ring atoms, in which one or more N, S and/or O atoms are present as heteroatoms and in which, in addition, one or two CH groups may be replaced by C═O. Unless defined otherwise, the heterocyclic rings preferably have 1-13 carbon atoms and 1-6 heteroatoms, in particular 3-9 carbon atoms and 1-4 heteroatoms. For example, heteroaromatic radicals such as 2- or 3-thienyl, 2- or 3-furyl, 2-, 3- or 4-pyridyl, pyrimidyl, pyrazolyl, pyrazolonyl, imidazolyl, triazinyl, pyrazinyl, thiazolyl, indolyl, quinolyl, quinoxalinyl or isoquinolyl are suitable.

The Ar and Het groups described above are preferably unsubstituted or monosubstituted, disubstituted or trisubstituted, possible substituents in principle being all substituents so long as they do not have a toxic effect on the compounds as a whole. The substituents defined as Sub² are preferably suitable and accordingly preference is given to the following substituents: halogen, in particular F or Cl, hydroxyl, amino, cyano, C₁-C₄-alkyl or C₁-C₄-alkoxy, COR⁵, COOR⁵, OAr, OHet, —(CR⁵R⁶)ₙ—Ar or —(CR⁵R⁶)ₙ-Het, —(CR⁵R⁶)ₙ—NR⁵R⁶, CONR⁵R⁶, CN, O═S(—R⁵)═O, O═S(—OR⁵)═O, O═S(—NR⁵R⁶)═O or R⁵OP(—OR⁶)═O.

It is furthermore also possible for a CH group in the ring system to be replaced by C═N—, i.e. the substituent is then ═N—R*, in which R* is the same as R⁵ or is —NH—Ar (see formula Ib).

R⁵ and R⁶ are then preferably H, C₁-C₄-alkyl, Ar or Het.

The following substituents are furthermore very particularly preferred: fluorine, chlorine, —COOH, alkoxy having up to 8 carbon atoms, —COO-alkyl having up to 8 carbon atoms, —CO-phenyl, —CO-aryl, —CO-Het, -quinoxalinyl or —CO—NH—R⁵. This list has a purely illustrative character and is in no way intended to be limiting.

In the above definitions, the groups Ar, Het, R⁵ and/or R⁶ may likewise in turn carry substituents as described above or below for these groups.

Particular preference is given to the use of the following compounds Ie to Ih, which conform to the formula I, but in which the radicals have the following preferred meaning:

Compounds of the sub-formula Ie which otherwise correspond to the compounds of the formula I, but in which

R¹ and R² are each, independently of one another, H,

alkyl having up to 20 carbon atoms, alkoxy up to 12 carbon atoms, in which, in addition, one or more hydrogen atoms may be substituted by Sub¹ and/or one or two CH₂ groups may be replaced by C═O,

Hal, OH, NO₂, —(CR⁵R⁶)ₙ—NR⁵R⁶, —(CR⁵R⁶)ₙ—N═CR⁵R⁶, —(CR⁵R⁶)ₙ—CR⁵═NR⁵, —(CR⁵R⁶)ₙ—NHCOR⁵ or —(CR⁵R⁶)ₙ—NHCOOR⁵,

R⁵ and R⁶ are each, independently of one another, as defined in the formula I, but preferably one of these radicals is H or alkyl having up to 7 carbon atoms and the other radical is then Ar or Het, each having up to 12 ring atoms, which may also preferably be substituted by 1-3 radicals,

and n, R³ and R⁴ are as defined in the formula I.

Compounds of the sub-formula If which conform to the formula I in which

R¹ is fluorine or chlorine, alkylhalogens, such as —CH₂Hal, —CHHal₂ or CHal₃, hydroxyl, nitro, —(CR⁵R⁶)ₙ—NR⁵R⁶, —(CR⁵R⁶)ₙ—N═CR⁵R⁶, —(CR⁵R⁶)ₙ—CR⁵═NR⁵, —(CR⁵R⁶)ₙ—NHCOR⁵ or —(CR⁵R⁶)ₙ—NHCOOR⁵,

R² is H, alkyl or alkoxy, each having up to 10 carbon atoms, halogen or alkylhalogen, nitro, hydroxyl, amino, C₁-C₇-aminoalkyl, C₁-C₇-diaminoalkyl, —(CR⁵R⁶)N—NHCOR⁵ or —(CR⁵R⁶)ₙ—NHCOOR⁵,

R⁵ and R⁶ are each, independently of one another, as defined in the formula I, but preferably one of these radicals is H or alkyl having up to 7 carbon atoms and the other radical is then Ar or Het, each having up to 12 ring atoms, which may also preferably be substituted by 1-3 radicals,

and n, R³ and R⁴ are as defined in the formula I.

Compounds of the sub-formula Ig which conform to the formula I in which

R³ and R⁴ are each, independently of one another, H, alkyl having up to 20 carbon atoms or alkoxy having up to 12 carbon atoms, in each of which, in addition, one or more hydrogen atoms may be substituted by Sub¹ and/or one or two CH₂ groups may be replaced by C═O,

hydroxyl, Hal, alkylhalogen, nitro, COR⁵, COOR⁵, CONR⁵R⁶, —(CR⁵R⁶)ₙ—NR⁵R⁶, —(CR⁵R⁶)ₙ—N═CR⁵R⁶, —(CR⁵R⁶)ₙ—CR⁵═NR⁵, —(CR⁵R⁶)ₙ—NHCOR⁵ or —(CR⁵R )ₙ—NHCOOR¹,

R⁵ and R⁶ are each, independently of one another, as defined in the formula I, but preferably one of these radicals is H or alkyl having up to 7 carbon atoms and the other radical is then Ar or Het, each having up to 12 ring atoms, which may also preferably be substituted by 1-3 radicals,

and n, R¹ and R² are as defined in the formula I.

Compounds of the sub-formula Ih which conform to the formula I in which

R³ is fluorine or chlorine,

alkylhalogen, in particular —CH₂Hal, —CHHal₂ or CHal₃, hydroxyl, nitro, —(CR⁵R⁶)ₙ—NR⁵R⁶, —(CR⁵R⁶)ₙ—N═CR⁵R⁶, —(CR⁵R⁶)ₙ—CR⁵═NR⁵, —(CR⁵R⁶)ₙ—NHCOR⁵ or —(CR⁵R⁶)ₙ—NHCOOR⁵,

R⁴ is H,

alkyl or alkoxy, each having up to 10 carbon atoms, halogen or alkylhalogen, nitro, hydroxyl, amino, C₁-C₇-aminoalkyl, C₁-C₇-diaminoalkyl, —(CR⁵R⁶)ₙ—NHCOR⁵, —(CR⁵R⁶)ₙ—NHCOOR⁵—(CR⁵R⁶)—NR⁵R⁶, COR⁵, COOR⁵ or CONR⁵R⁶,

R⁵ and R⁶ are each, independently of one another, as defined in the formula I, but preferably one of these radicals is H or alkyl having up to 7 carbon atoms and the other radical is then Ar or Het, each having up to 12 ring atoms, which may also preferably be substituted by 1-3 radicals,

and n, R¹ and R² are as defined in the formula I.

In a particularly preferred embodiment of this invention, use is also made of compounds of the sub-formulae Ii or Ij

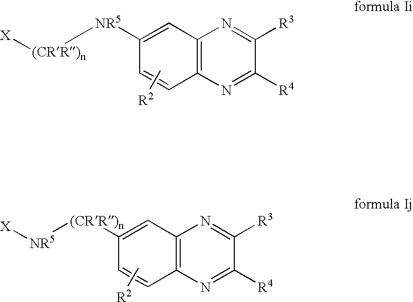

in which R², R³, R⁴, R⁵ R′, R″ and n are as defined in the formula I, and X are Ar or Het with the definitions given therefor. X is preferably an unsubstituted or monosubstituted to trisubstituted aromatic or heteroaromatic ring having from 5 to 7 ring atoms.

Compounds of the sub-formulae Ik and Il are furthermore particularly suitable for the use according to the invention:

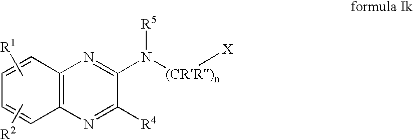

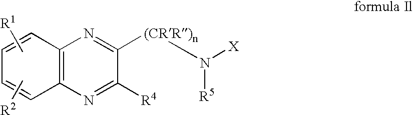

in which R², R¹, R⁴, R⁵, R′, R″ and n are as defined in the formula I, and X are Ar or Het with the definitions given therefor. X is preferably an unsubstituted or monosubstituted to trisubstituted aromatic or heteroaromatic ring having from 5 to 7 ring atoms.

The following compounds Im to In are likewise, for example, particularly preferably:

Im compounds which otherwise conform to the formula Il in which R⁴ are then phenyl or —NH-phenyl;

In compounds which otherwise conform to the formula I in which R⁴ is —NR⁵R⁶, in which R⁵ is a quinoxaline ring and R⁶ is Ar or Het.

If the compounds of the formula I and of the sub-formulae Ia to In contain the groups Ar and/or Het, these are preferably monosubstituted to trisubstituted. Table 1 lists the preferred substituents with the preferred positions on the ring using the example of the phenyl ring, which is particularly preferably selected. Especial preference is also given to compounds of the sub-formulae Ii to Il which contain a phenyl ring as X.

TABLE 1

| R | m | Position | 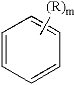 |
|---|---|---|---|
| CH₃ | 1-3 | o/m/p; m/m/p; o/p; m/m | |
| C₂H₅ | 1-3 | ″ | |
| C₃H₇ | 1-3 | ″ | |
| C₄H₉ | 1-3 | ″ | |
| C₅H₁₁ | 1-3 | ″ | |
| C₁₈H₃₇ | 1-3 | ″ | |
| OCH₃ | 1-3 | o/m/p; m/m/p; o/p; m/m | |
| OC₂H₅ | 1-3 | ″ | |
| OC₃H₇ | 1-3 | ″ | |
| OC₄H₉ | 1-3 | ″ | |
| OC₅H₁₁ | 1-3 | ″ | |
| OC₁₈H₃₇ | 1-3 | ″ | |
| OCOCH₃ | 1-2 | o; m; p; o/p | |
| OCOC₂H₅ | 1-2 | ″ | |
| OCOC₃H₇ | 1-2 | ″ | |
| OCOC₄H₉ | 1-2 | ″ | |
| OCOC₅H₁₁ | 1-2 | ″ | |
| OCOC₁₈H₃₇ | 1-2 | ″ | |
| OH | 1-3 | o/m/p; m/m/p; m/m | |
| F | 1-2 | o; p; o/p | |
| Cl | 1-2 | o; p; o/p | |
| CF₃ | 1 | o; m; p | |
| NO₂ | 1-3 | m/; m/m; o/o/p | |
| NHCOR⁵ | 1 | p | |
| NHCOOR⁵ | 1 | p | |
| COR⁵ | 1-2 | o; p; o/p | |
| COOR⁵ | 1-2 | o; p; o/p | |
| CONR⁵R⁶ | 1-2 | o; p; o/p | |
| CN | 1 | p | |
| O═S(OR⁵)═O | 1 | p | |
| O═S(R⁵)═O | 1 | p | |
| O═S(NR⁵R⁶)═O | 1 | p | |

Preference is furthermore given to compounds of the formula I in which R¹ and R² are each H, and R³ and R⁴ then have the following meanings:

R³ is —CH═CH-quinoxalin-2-yl and R⁴ is H;

R³ is —CH₂—CH(NHR⁵)COOR⁶ and R⁴ is H;

R³ is —CH₂-2-[1,3-(2-alkyl)imidazol-4-one] and R⁴ is H;

R³ is —CH₂—CH₂—PO(OR⁷)₂ and R⁴ is —CH₂—CH(NH₂)COOH;

R³ is —CH₂—CO—CH═CHCOOR⁵ and R⁴ is H;

R³ is —O-phenyl (substituted by O-alkyl) and R⁴ is H;

R³ is —O-phenyl, substituted by —CO—NH—CH(CH₂COOR⁵)—CH₂—CH₂—COOR⁵ and R⁴ is H;

The following compounds of the formulae II and III are likewise particularly preferred:

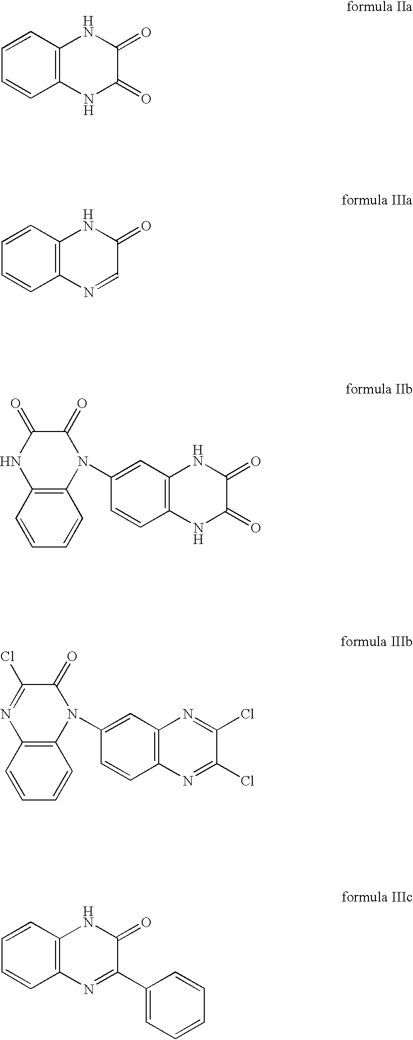

The majority of the compounds of the formula I, II and/or III are known and can be prepared by generally known methods for the preparation of quinoxaline derivatives (for example described in standard works, such as Beilstein or Houben-Weyl). A process for the preparation of secondary quinoxalin-2-ylmethylamines is also described, for example, in the patent specification DD 281 380, in which halomethylquinoxalines are reacted with amino compounds at elevated temperature in inert solvents.

The formulae I, II and/or III also cover some compounds which are novel.

The invention therefore also relates to the novel compounds of the formulae indicated above. In particular, the invention relates to the novel compounds of the formulae Ik and Il, and the following compounds:

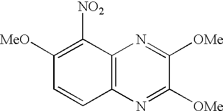

5-nitro-2,3,6-trimethoxyquinoxaline;

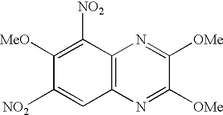

2,3,7-trimethoxy-6,8-dinitroquinoxaline;

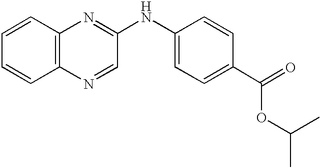

isopropyl N-(2-quinoxalinyl)-4-aminobenzoate;

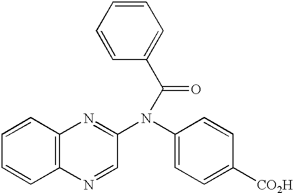

N,N-benzoyl-(2-quinoxalinyl)₄-aminobenzoic acid;

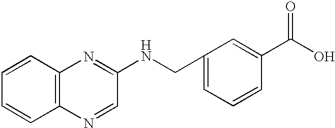

N-(2-quinoxalinyl)-4-aminomethylbenzoic acid;

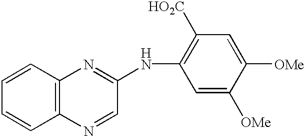

N-(2-quinoxalinyl)-2-amino-4,5-dimethoxybenzoic acid;

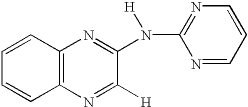

N-(2-quinoxalinyl)-2-aminopyrimidine;

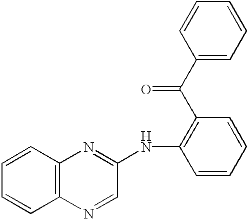

N-(2-quinoxalinyl)-2-aminobenzophenone;

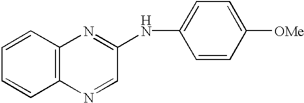

N-(2-quinoxalinyl)-4-aminoanisole;

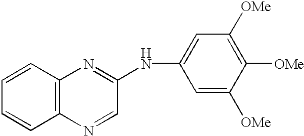

N-(2-quinoxalinyl)-3,4,5-trimethoxyaniline;

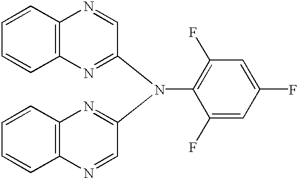

N-[bis(2-quinoxalinyl)]-2,4,6-trifluoroaniline;

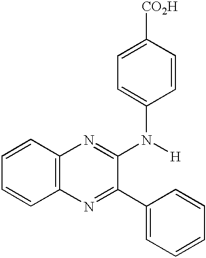

N-[2-(3-phenyl)quinoxalinyl]-4-aminobenzoic acid;

which are also particularly preferably used.

triazine and aminotriazine radicals are also preferred as substituents on the quinoxaline structure. Correspondingly, the following examples and derivatives thereof with radicals substituted in accordance with the invention also belong to the compounds which are preferred in accordance with the invention:

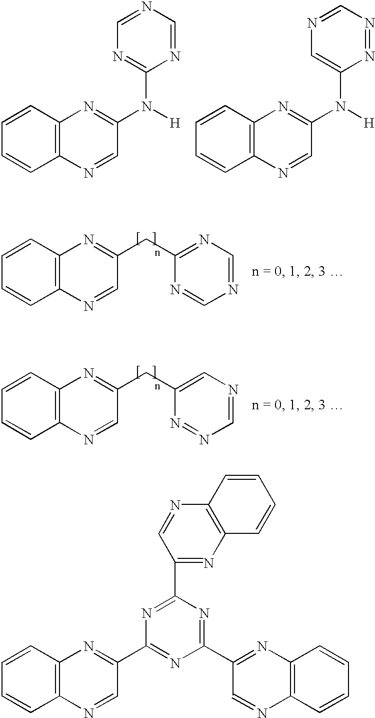

The following, already known quinoxaline derivatives are likewise preferably employed for the use according to the invention. These compounds can be prepared by methods known from the literature which are familiar to the person skilled in the art:

N-(2-quinoxalinyl)-2,4-dichloroaniline

N-[bis(3-fluoroquinoxalin-2-yl)]-2-aminopyrimidine

2-chloro-3-phenylquinoxaline

3-phenylquinoxalin-2-one

6-chloro-2,3-dimethylquinoxaline

2-chloromethylquinoxaline

diethyl N-(2-quinoxalinyl)]-4-aminobenzoylglutamate

2-chloroquinoxaline

3,6-dichloro-1-(2,3,7-trichloroquinoxalin-6-yl) quinoxalin-2(1H)-one

1,2,3,4-tetrahydro-1-(1,2,3,4-tetrahydro-2,3-dioxoquinoxalin-6-yl) quinoxalin-2,3-dione

quinoxalin-2-one

N-(2-quinoxalinyl)-4-aminobenzoic acid

2,3-dimethoxy-6-aminoquinoxaline

2,3-dimethoxy-6-nitroquinoxaline

2,3,7-trimethoxyquinoxaline

6-chloro-2,3-dimethoxyquinoxaline.

The quinoxaline derivatives according to the invention are outstandingly suitable as UV filter substances. As already mentioned, the quinoxalines can be designed synthetically in such a way that the presence of an additional chromophoric group gives rise to UV-absorbent properties both in the UV-A and in the UV-B region. Broad-band protection can thus be achieved. Furthermore, the solubility of the substances in water or cosmetic oils can be influenced through the choice of substituents.

If desired, the quinoxaline derivatives can also be combined with any desired UV filter substances, which results in an improvement in the protective performance (SPF boost) through synergistic effects. Some UV-A and UV-B filter substances with which the quinoxalines according to the invention can preferably be combined are listed below. This selection is in no way intended to be limiting. The combination can by chemical reaction (Table 2) and/or physical combination with UV filters listed below.

TABLE 2

| | |
|---|---|
| 4-Aminobenzoic acid | see formula IV |
| Dimethoxyphenylglyoxalic acid | |
| 2,2′,4,4′-Tetrahydroxybenzophenone | |
| 3,3,5-trimethylcyclohexyl salicylate | see formula V |
| 2,2′-Dihydroxy-4-methoxybenzophenone | |
| 5-Methyl-2-(1-methylethyl) 2-aminobenzoate | |
| 3-Imidazol-4-ylacrylic acid and ethylester | |
| 4-Isopropylbenzylsalicylate | |
| 1-(4′-Isopropylphenyl)-3-phenylpropane- | |
| 1,3-dione | |
| 3-Benzylidenebornan-2-one (3- | |
| benzylidenecamphor) | |
| 3-(4′-Methyl)benzylidenebornan-2-one | see formula VI |
| 3-(4′-Trimethylammonium) | |
| benzylidenebornan-2-one | |
| methylsulfate | |
| 1-(4′-tert-Butylphenyl)-3-(4- | see formula VII |
| methoxyphenyl)propane- | |
| 1,3-dione (for example Eusolex ® 9020) | |
| 3-(4′ Sulfo)benzylidenebornan-2-one | |
| and salts | |
| 2-Hydroxy-4-methoxybenzophenone-5- | |
| sulfonic acid/Na salt | |
| 2-Ethylhexyl salicylate | |
| 2-Hydroxy-4-methoxy-4′- | |
| methylbenzophenone | |
| 2-Hydroxy-4-methoxybenzophenone | |
| 2-Isoamyl 4-methoxycinnamate | |
| 2-Ethylhexyl 4-methoxycinnamate | |
| 1-Glyceryl 4-aminobenzoate | |

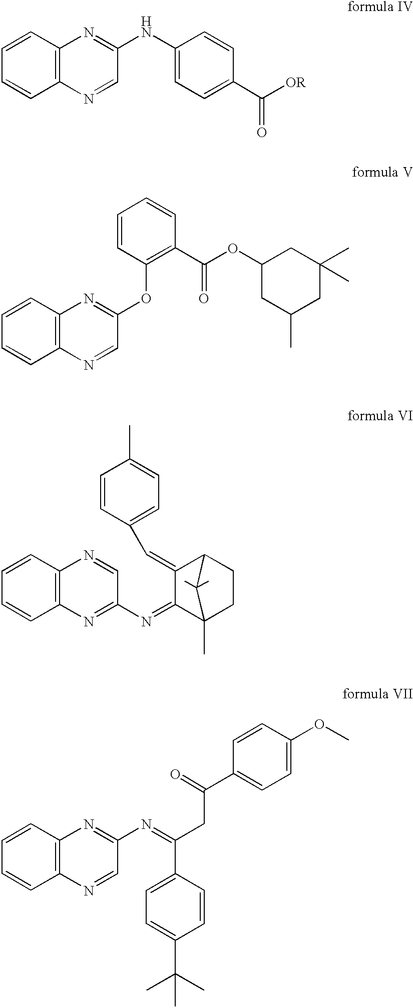

In principle, all UV filters are suitable for a combination. Particular preference is given to UV filters whose physiological acceptability has already been demonstrated. Substances are known from the specialist literature both for UV-A and UV-B filters, for example

benzylidenecamphor derivatives, such as

3-(4′-methylbenzylidene)-dl-camphor (for example Eusolex® 6300),

3-benzylidenecamphor (for example Mexoryl® SD),

polymers of N-{(2 and 4)-[(2-oxoborn-3-ylidene)methyl]benzyl}acrylamide (for example Mexoryl® SW),

N,N,N-trimethyl-4-(2-oxoborn-3-ylidenemethyl)anilinium methylsulfate (for example Mexoryl® SK) or

α-(2-oxoborn-3-ylidene)toluene-4-sulfonic acid (for example Mexoryl® SL),

benzoyl- or dibenzoylmethanes, such as

1-(4-tert-butylphenyl)-3-(4-methoxyphenyl)propane-1,3-dione (for example Eusolex® 9020) or

4-isopropyldibenzoylmethane (for example Eusolex® 8020),

benzophenones, such as

2-hydroxy-4-methoxybenzophenone (for example Eusolex® 4360) or

2-hydroxy-4-methoxybenzophenone-5-sulfonic acid and its sodium salt (for example Uvinul® MS-40),

methoxycinnamic acid esters, such as

octyl methoxycinnamate (for example Eusolex® 2292),

isopentyl 4-methoxycinnamate, for example as a mixture of the isomers (for example Neo Heliopan® E 1000),

salicylate derivatives, such as

2-ethylhexyl salicylate (for example Eusolex® OS),

4-isopropylbenzyl salicylate (for example Megasol®) or

3,3,5-trimethylcyclohexyl salicylate (for example Eusolex® HMS),

4-aminobenzoic acid and derivatives, such as

4-aminobenzoic acid,

2-ethylhexyl 4-(dimethylamino)benzoate (for example Eusolex® 6007),

ethoxylated ethyl 4-aminobenzoate (for example Uvinul® P25),

and further substances, such as

2-ethylhexyl 2-cyano-3,3-diphenylacrylate (for example Eusolex® OCR),

2-phenylbenzimidazole-5-sulfonic acid and its potassium, sodium and triethanolamine salts (for example Eusolex® 232),

3,3′-(1,4-phenylenedimethylene)bis(7,7-dimethyl-2-oxobicyclo[2.2.1]-hept-1-ylmethanesulfonic acid and its salts (for example Mexoryl® SX) and

2,4,6-trianilino-(p-carbo-2′-ethylhexyl-1′-oxy)-1,3,5-triazine (for example Uvinul® T 150).

The compounds mentioned in the list should only be regarded as examples. It is of course also possible to use other UV filters. These organic UV filters are generally incorporated into cosmetic preparations in an amount of from 0.5 to 10 percent by weight, preferably 1-8%.

Further suitable organic UV filters are, for example,

2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),

2-ethylhexyl 4,4′-[(6-[4-((1,1-dimethylethyl)aminocarbonyl)phenylamino]-1,3,5-triazine-2,4-diyl)diimino]bis(benzoate) (for example UV-Asorb® HEB),

α-(trimethylsilyl)-ω-[trimethylsilyl)oxy]poly[oxy(dimethyl [and approximately 6% of methyl[2-[p-[2,2-bis(ethoxycarbonyl]vinyl]phenoxy]-1-methyleneethyl] and approximately 1.5% of methyl[3-[p-[2,2-bis(ethoxy-carbonyl)vinyl)phenoxy)propenyl) and from 0.1 to 0.4% of (methylhydrogen]silylene]] (n≈60) (CAS No. 207 574-74-1)

2,2′-methylenebis(6-(2H-benzotriazol-2-yl)-4-(1,1,3,3-tetramethylbutyl)-phenol) (CAS No.103 597-45-1)

2,2′-(1,4-phenylene)bis)-1H-benzimidazole-4,6-disulfonic acid, monosodium salt) (CAS No.180 898-37-7) and

2,2′-(1,4-phenylene)bis)-1H-benzimidazole-5,5′-monosulfonic acid, monosodium salt

2,4-bis{[4-(2-ethylhexyloxy)-2-hydroxy]phenyl}-6-(4-methoxyphenyl)-1,3,5-triazine (CAS No.103 597-45-, 187 393-00-6).

These organic UV filters are generally incorporated into cosmetic preparations in an amount of from 0.5 to 20 percent by weight, preferably 1-15%. They are usually present in formulations according to the invention in weight ratios of from 15:1 to 1:15, preferably from 10:1 to 1:10 and particularly preferably from 5:1 to 1:5 to the quinoxaline derivatives of the formulae I, II or III.

Preferred compounds having UV-filtering properties are 3-(4′-methylbenzylidene)dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and its potassium, sodium and triethanolamine salts.

Conceivable inorganic UV filters are, for example, those from the group consisting of titanium dioxides, such as, for example, coated titanium dioxide (for example Eusolex® T-2000, Eusolex® T-AQUA), zinc oxides (for example Sachtotec®), iron oxides and also cerium oxides. Titanium oxide and zinc oxide are preferably in the form of micronised inorganic pigments. These inorganic UV filters are generally incorporated into cosmetic preparations in an amount of from 0.5 to 20 percent by weight, preferably 2-10%. They are usually present in formulations according to the invention in weight ratios of from 25:1 to 1:25, preferably from 10:1 to 1:10 and particularly preferably from 5:1 to 1:5 to the quinoxaline derivatives of the formulae I, II or III.

The use according to the invention of the quinoxaline derivatives has a further, interesting and advantageous aspect. The combination with other UV filters often results in photostabilisation of this other substance by the quinoxaline derivative.

It is known that some UV filters which have advantageous light-protection filter properties per se have the great disadvantage of a certain instability to UV radiation.

For example, the dibenzoylmethane derivatives, such as Eusolex 9020 (4-t-butyl-4′-methoxydibenzoylmethanes), are substances which are exposed to photochemical decomposition. The photochemical decomposition of this class of compounds follows a Norrish type I acyl cleavage. The reaction products formed in the process are no longer available as light-protection filter substances. Although some proposed solutions have already been indicated in the prior art, there nevertheless continues to be a demand for simple and effective ways of effectively countering this photolytic decomposition.

The quinoxaline derivatives described here are outstandingly suitable for this purpose, in particular combination with Eusolex 9020 results in considerably improved photostabilisation of the substance.

In accordance with the invention, the UV light-protection filters described here can in each case be used alone or naturally also in combination, which is preferred, in sunscreens. They can be combined with UV-B/A chromophores, for example all filters approved and known worldwide, for improving the protective performance (SPF boost) through synergistic effects. They can preferably be employed in combination both with inorganic and with organic UV-A and UV-B filters or mixtures thereof.

Through combination of one or more compounds of the formula I with further UV filters, the protective action against harmful effects of UV radiation can be optimised.

It is furthermore possible and advantageous to combine the preparations according to the invention with antioxidants. A combination of this type then exhibits both a protective action as antioxidant and also against burns due to UV radiation. A protective action against oxidative stress or against the action of free radicals can thus also be achieved.

The invention therefore also relates to the use of a compound of the formula I according to claim 1 in combination with antioxidants in cosmetic or pharmaceutical preparations.

The present invention therefore also relates to cosmetic and pharmaceutical preparations which comprise one or more of the compounds of the formulae I, II and/or III, optionally in combination with further light-protection agents or antioxidants.

The invention also relates to a method for protecting the skin and natural or sensitised hair against solar radiation, in which an effective amount of at least one compound of the formula I, II and/or III in a cosmetic preparation is applied to the skin or the hair. “Sensitised hair” is taken to mean hair which has been subjected to permanent-wave treatment, a dyeing process or a bleaching process.

The filters according to the invention for protection against UV-A and UV-B radiation can in each case be incorporated into cosmetic preparations in concentrations of from 0.1 to 20% by weight, preferably from 1 to 15% by weight. In this way, it is possible to prepare preparations in which up to 100% of the light-protection filters employed are the UV filters described here. These are substances which are dissolved, dispersed or emulsified in a simple manner in water and oils, depending on the substituents on the skeleton.

The preparations according to the invention may in addition comprise further conventional skin-protecting or skincare active ingredients. These may in principle be any active ingredients known to the person skilled in the art.

Particularly preferred active ingredients are pyrimidinecarboxylic acids and/or aryl oximes, as well as coumaranone derivatives.

Pyrimidinecarboxylic acids occur in halophilic microorganisms and play a role in osmoregulation of these organisms (E. A. Galinski et al., Eur. J. Biochem., 149 (1985) pages 135-139). Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid and their derivatives. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoin and ectoin derivatives, such as hydroxyectoin, can advantageously be used in medicaments. In particular, hydroxyectoin can be employed for the preparation of a medicament for the treatment of skin diseases. Other areas of application of hydroxyectoin and other ectoin derivatives are typically in areas in which, for example, trehalose is used as additive. Thus, ectoin derivatives, such as hydroxyectoin, can be used as protectant in dried yeast and bacteria cells. Pharmaceutical products, such as non-glycosylated, pharmaceutical active peptides and proteins, for example t-PA, can also be protected with ectoin or its derivatives.

Of the cosmetic applications, particular mention should be made of the use of ectoin and ectoin derivatives for the care of aged, dry or irritated skin. Thus, European Patent Application EP-A-0 671 161 describes, in particular, that ectoin and hydroxyectoin are employed in cosmetic preparations, such as powders, soaps, surfactant-containing cleansing products, lipsticks, rouge, makeups, care creams and sunscreen preparations.

Preference is given here to the use of a pyrimidinecarboxylic acid of the following formula,

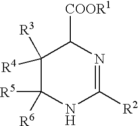

in which R¹ is a radical H or C1-8-alkyl, R² is a radical H or C1-4-alkyl, and R³, R⁴, R⁵ and R⁶ are each, independently of one another, a radical from the group consisting of H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoin ((S)-1,4,5,6-tetrahydro-2-methyl-4-pyrimidinecarboxylic acid) and hydroxyectoin ((S, S)-1,4,5,6-tetrahydro-5-hydroxy-2-methyl-4-pyrimidinecarboxylic acid). The preparations according to the invention preferably comprise pyrimidinecarboxylic acids of this type in amounts of up to 15% by weight.

Of the aryl oximes, preference is given to the use of 2-hydroxy-5-methyllaurophenone oxime, which is also known as HMLO, LPO or F5. Its suitability for use in cosmetic compositions is disclosed, for example, in DE-A-41 16 123. Preparations which comprise 2-hydroxy-5-methyllaurophenone oxime are accordingly suitable for the treatment of skin diseases which are accompanied by inflammation. It is known that preparations of this type can be used, for example, for the therapy of psoriasis, various forms of eczema, irritative and toxic dermatitis, UV dermatitis and further allergic and/or inflammatory diseases of the skin and integumentary appendages. Preparations according to the invention which, in addition to the compound of the formula I, additionally comprise an aryl oxime, preferably 2-hydroxy-5-methyllaurophenone oxime, exhibit surprising antiinflammatory suitability. The preparations here preferably comprise from 0.01 to 10% by weight of the aryl oxime, it being particularly preferred for the preparation to comprise from 0.05 to 5% by weight of aryl oxime.

Coumaranone derivatives which may advantageously be present in the preparations according to the invention are compounds of the formula

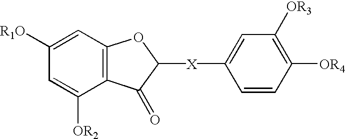

in which —X— is a single bond, —CH₂—, ═CH—, —C(O)—, ═C(OR⁵)—, —C(NR⁵)—, —CH(NR⁵R⁶)— or —CH(OR⁵)—, and R¹, R², R³, R⁴, R⁵, and R⁶ may be identical or different and are, independently of one another,

H

straight-chain or branched C₁- to C₁₂-alkyl and/or alkylcarbonyl groups,

straight-chain or branched C₃- to C₁₂-alkenyl and/or -alkenylcarbonyl groups,

straight-chain or branched C₁- to C₁₂-hydroxyalkyl groups, in which the hydroxyl group may be bonded to a primary or secondary carbon atom of the chain, and furthermore the alkyl chain may also be interrupted by oxygen,

C₃- to C₁₀-cycloalkyl and/or cycloalkylcarbonyl groups and C₃- to C₁₂-cycloalkenyl and/or cycloalkenylcarbonyl groups, in which each of the rings may also be bridged by —(CH₂)ₙ— groups, where n=from 1 to 3,

aryl and/or arylcarbonyl groups,

heteroaryl and/or heteroarylcarbonyl groups, where these groups may be substituted by alkyl, hydroxyl, alkoxy, amino, mono- and dialkylamino, sulfonic acid, carboxyl and/or halogen groups,

mono- and/or oligoglycosyl radicals,

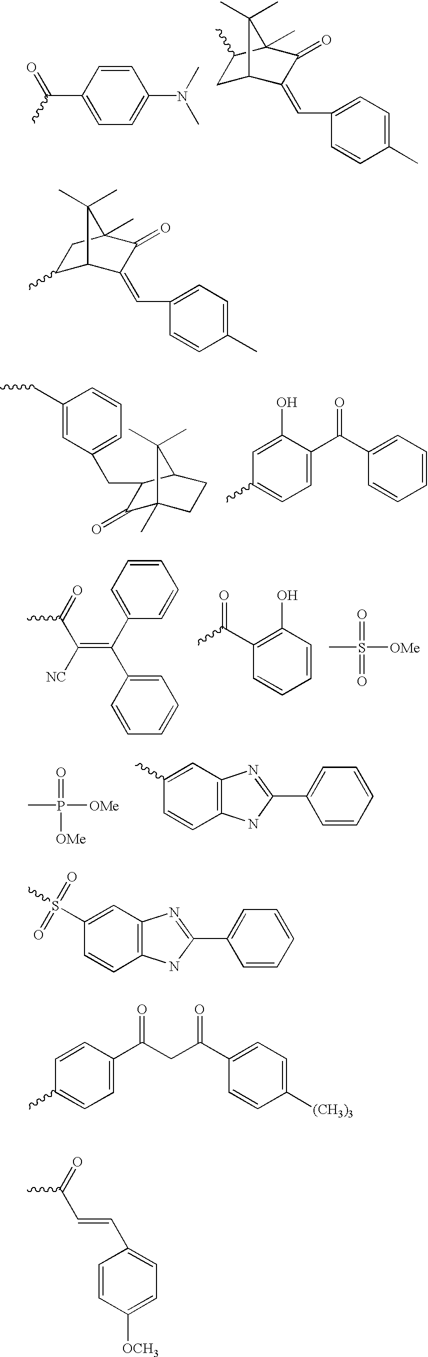

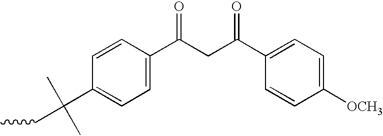

in which Me is a proton or an alkali metal ion, in particular a sodium or potassium ion.

The radicals can thus be bonded to the parent structure as ethers or as esters. Compounds of this type are described in the German patent application DE 10003785.2. Compositions which comprise coumaranone derivatives of this type have a particularly gentle action on the skin, which these compounds exhibit an antioxidant and free-radical-scavenging action.

The parent structure 4,6,3′,4′-tetrahydroxybenzyl-3-coumaranone has very good properties. This compound conforms to the above formula, in which X=—CH₂—, and R¹=R²=R³=R⁴=H. The solubility of this compound in water can be improved by selecting, for example, the radicals R¹, R², R³ and R⁴ as sulfate or phosphate groups. A mixture of mono-, di- and trisulfate, which is referred to below as “sulfated coumaranone”, is particularly suitable. Particular emphasis should be placed on the trisulfate (X=—CH₂—; R¹=R³=R⁴=SO₃Me, R²=H), which is represented by the following formula.

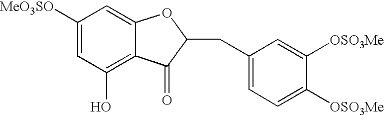

All compounds or components which can be used in the preparations are either known and commercially available or can be synthesised by known processes.

The cosmetic and pharmaceutical preparations comprising light-protection agents are generally based on a vehicle which comprises at least one oil phase. However, preparations on a purely aqueous basis are also possible in the case of the use of compounds having hydrophilic substituents. Accordingly, oils, oil-in-water and water-in-oil emulsions, creams and pastes, lip protection compositions or fat-free gels are possible.

Sunscreen preparations of this type can thus be in liquid, pasty or solid form, for example as water-in-oil creams, oil-in-water creams and lotions, W/O/W systems or O/W/O systems, aerosol foam creams, gels, oils, fat sticks, powders, sprays or alcoholic/aqueous lotions. They can furthermore also be formulated as micronised systems or as PIT (phase inversion temperature) emulsions.

Conventional oil components in cosmetics are, for example, paraffin oil, glyceryl stearate, isopropyl myristate, diisopropyl adipate, acetylstearyl 2-ethylhexanoate, hydrogenated polyisobutene, Vaseline, caprylic acid/capric acid triglycerides, microcrystalline wax, lanolin, mineral oils, mineral waxes, esters of fatty acids with alcohols having a low carbon number, for example with isopropanol, propylene glycol or glycerol, alkyl benzoates, silicone oils, such as dimethylpolysiloxanes, diethylpolysiloxanes, diphenylpolysiloxanes, and stearic acid.

The preparations may comprise cosmetic adjuvants which are usually used in preparations of this type, such as, for example, thickeners, softeners, moisturisers, surface-active agents, emulsifiers, preservatives, antifoams, perfumes, fats and waxes, lanolin, propellants, stabilisers, antioxidants, bactericides, dyes and/or pigments which colour the composition itself or the skin, and other ingredients usually used in cosmetics.

Suitable emulsifiers are preferably known W/O and in addition also O/W emulsifiers, such as, for example, polyglycerol esters, sorbitan esters or partially esterified glycerides. Typical examples of fats are glycerides; waxes which should be mentioned are, inter alia, beeswax, carnauba wax, paraffin wax or microwaxes, optionally in combination with hydrophilic waxes. Stabilisers which can be employed are metal salts of fatty acids, such as, for example: magnesium stearate, aluminium stearate and/or zinc stearate. Suitable thickeners are, for example, crosslinked polyacrylic acids and derivatives thereof, polysaccharides, in particular xanthan gum, guar-guar, agar-agar, alginates and tyloses, carboxymethylcellulose and hydroxyethylcellulose, furthermore fatty alcohols, monoglycerides and fatty acids, polyacrylates, polyvinyl alcohol and polyvinylpyrrolidone. It is furthermore also possible to add plant extracts, protein hydrolysates and vitamin complexes.

Antioxidants which can be used are, for example, amino acids, imidazoles, peptides, carotenoids, α-hydroxyacids, unsaturated fatty acids, vitamin A, C and/or E, and suitable derivatives of these substances.

There are many proven substances known from the specialist literature which can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotenoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof), and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents, (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordohydroguaiaretic acid, trihydroxybutyrophenone, quercetin, uric acid and derivatives thereof, mannose and derivatives thereof, zinc and derivatives thereof (for example ZnO, ZnSO4), selenium and derivatives thereof (for example selenomethionine), stilbenes and derivatives thereof (for example stilbene oxide, transstilbene oxide).

Mixtures of antioxidants are likewise suitable for use in the cosmetic preparations according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004).

The quinoxaline derivatives of the formulae I, II or III are usually present in the formulations according to the invention in weight ratios to the antioxidants of from 10,000:1 to 1:5, preferably from 500:1 to 1:2 and particularly preferably from 50:1 to 1:1.

The preparations according to the invention may comprise vitamins as further ingredients. The cosmetic preparations according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine, (vitamin B₆), panthothenic acid, biotin, folic acid and cobalamine (vitamin B₁₂), particularly preferably vitamin A palmitate, vitamin C, DL-α-tocopherol, tocopherol E acetate, nicotinic acid, panthothenic acid and biotin.

The aqueous phase of the preparations according to the invention optionally advantageously comprise alcohols, diols or polyols having a low carbon number, and ethers thereof, preferably ethanol, isopropanol, propylene glycol, glycerol, ethylene glycol, ethylene glycol monoethyl or monobutyl ether, or analogous products, furthermore alcohols such as ethanol, isopropanol, 1,2-propanediol and, in particular, one or more thickeners, such as, for example, silicon dioxide, aluminium silicates, polysaccharides or derivatives thereof, for example hyaluronic acid, xanthan gum or hydroxypropylmethylcellulose, or a polyacrylate from the group consisting of the so-called Carbopols.

Conventional film formers are, for example, hydrocolloids, such as chitosan, microcrystalline chitosan or quaternary chitosan, polyvinylpyrrolidone, vinylpyrrolidone-vinyl acetate copolymers, polymers from the acrylic acid series, quaternary cellulose derivatives and similar compounds. Examples of suitable preservatives are formaldehyde solutions, p-hydroxybenzoate or sorbic acid. Suitable pearlescent agents are, for example, glycoldistearic acid esters, such as ethylene glycol distearate, but also fatty acids and fatty acid monoglycol esters. Dyes which can be used are the substances which are suitable and approved for cosmetic purposes, as compiled, for example, in the publication “Kosmetische Färbemittel” [Cosmetic Colorants] by the Dye Commission of the German Research Society, published in Verlag Chemie, Weinheim, 1984. These dyes are usually employed in concentrations of from 0.001 to 0.1% by weight, based on the mixture as a whole.

If desired, the sunscreens according to the invention may also comprise one or more chemical substances having self-tanning properties.

Chemical substances having self-tanning properties which can be employed are all natural and synthetic substances known to the person skilled in the art which are suitable for the preparation of cosmetic preparations. These may be either vegetable extracts or synthetic self-tanning agents, such as, for example, dihydroxyacetone or α-ketols.

If the composition according to the invention is intended to protect natural or sensitised hair against sunlight, it can be in the form of a rinse-out shampoo, lotion, gel or emulsion, the preparation in question being applied before or after shampooing, before or after colouring or bleaching or before or after permanent waving; or the composition is in the form of a lotion or gel for styling and treatment, in the form of a lotion or gel for brushing or blow-waving, in the form of a hair lacquer, hair spray, aerosol foam cream, permanent waving composition, dye or bleach for the hair. In addition to the light-protection filters according to the invention (VIS and/or IR filters) or the combination of light-protection filters according to the invention, this composition may also comprise various adjuvants used in this type of composition, such as surfactants, thickeners, polymers, softeners, preservatives, foam stabilisers, electrolytes, organic solvents, silicone derivatives, oils, waxes, anti-grease agents, dyes and/or pigments which colour the composition itself or the hair, or other ingredients usually used for hair care.

Further typically cosmetic use forms are also lipsticks, lip-care sticks, mascara, eyeliner, eyeshadow, rouge, powder, emulsion and wax makeup, and sunscreen, pre-sun and after-sun preparations.

The light-protection filters according to the invention can be incorporated directly into cosmetic preparations without further preparatory measures.

These substances furthermore offer the great advantage of exhibiting no toxic or allergic reactions with the skin.

The light-protection preparations can advantageously comprise in accordance with the invention, as already described above, further UV filter substances, where the total amount of the filter substances is, for example, from 0.1% by weight to 30% by weight, preferably from 0.5 to 10% by weight, in particular from 1 to 6% by weight, based on the total weight of the preparations.

The preparations according to the invention can furthermore also be used as pharmaceutical compositions for preventative treatment of inflammation and allergies of the skin and also in certain cases for the prevention of certain types of cancer. The pharmaceutical composition according to the invention can be administered orally or topically. For oral administration, the pharmaceutical preparation is in the form of, inter alia, pastilles, gelatine capsules, coated tablets, as a syrup, solution, emulsion or suspension. Topical application is carried out, for example, as an ointment, cream, gel, spray, solution or lotion.

The cosmetic and pharmaceutical preparations according to the invention can be prepared with the aid of methods which are well known to the person skilled in the art.

The properties of compounds of the formulae I, II and/or III should likewise be regarded as positive for use in foods, as food supplements, as dietary agent or as functional food. The further explanations given for foods logically also apply to food supplements and functional foods.

The foods which can be enriched in accordance with the present invention with one or more of the compounds according to the invention include all materials which are suitable for consumption by animals or consumption by humans, for example vitamins and provitamins thereof, fats, minerals or amino acids. (The foods may be solid, but also liquid, i.e. in the form of a beverage). Foods which can be enriched with one or more compounds of the formulae I, II and/or III in accordance with the present invention are, for example, also foods which originate from a single natural source, such as, for example, sugar, unsweetened juice, squash or puree of a single plant species, such as, for example, unsweetened apple juice (for example also a mixture of different types of apple juice), grapefruit juice, orange juice, apple compote, apricot squash, tomato juice, tomato sauce, tomato puree, etc. Further examples of foods which can be enriched with one or more compounds of the formula I in accordance with the present invention are corn or cereals from a single plant species and materials produced from plant species of this type, such as, for example, cereal syrup, rye flour, wheat flour or oatbran. Mixtures of foods of this type are also suitable for being enriched in accordance with the present invention with one or more of the compounds according to the invention, for example multivitamin preparations, mineral mixtures or sweetened juice. As further examples of foods, mention may be made of food preparations, for example prepared cereals, biscuits, mixed drinks, foods prepared especially for children, such as yoghurt, diet foods, low-calorie foods or animal feeds.

The foods which can be enriched in accordance with the present invention with one or more compounds of the formulae I, II and/or III thus include all edible combinations of carbohydrates, lipids, proteins, inorganic elements, trace elements, vitamins, water or active metabolites of plants and animals.

The foods which can be enriched in accordance with the present invention with one or more compounds of the formulae I, II and/or III are preferably administered orally, for example in the form of meals, pills, tablets, capsules, powders, syrup, solutions or suspensions.

These enriched foods according to the invention can be prepared using techniques which are well known to the person skilled in the art.

Even without further comments, it is assumed that a person skilled in the art will be able to utilise the above description in its broadest scope. The preferred embodiments should therefore merely be regarded as descriptive disclosure which is absolutely not limiting in any way.

The following examples are intended to illustrate the present invention without limiting it. All amount data, proportions and percentages are, unless stated otherwise, based on the weight and the total amount or the total weight of the preparations.

The complete disclosure content of all applications and publications mentioned above and below are incorporated into this application by way of reference.

### PREPARATION EXAMPLES

### Example A

Preparation of isopropyl N-(2-quinoxalinyl)-4-aminobenzoate

1 mmol of 2-chloroquinoxaline and 1.5 mmol of isopropyl 4-aminobenzoate are dissolved in 5 ml of n-propanol and refluxed at a temperature of 120° C. for 4 hours. The solid which precipitates during the reaction is, after cooling, separated off by filtration and recrystallised from ethyl acetate using activated carbon, giving 91% of the compound isopropyl N-(2-quinoxalinyl)-4-aminobenzoate as a yellowish powder.

Characterisation:

| IR (KBr): | | |
|---|---|---|
| Wavenumber | 3333 | (ν(N—H), NH) |
| [cm − 1] = | | |
| | 3155 | (ν(C—H), Ph) |
| | 1720 | (ν(C═O), CO₂R) |
| | 1613 | (ν(C═C), Ph) |
| | 1551 | (ν(C═C), Ph) |
| | 1290 | ((skeleton), Ph + CO) |
| | 760 | (γ(C—H), Ph) |
| | 699 | (γ(C—H), Ph) |
| MS: | | |
| m/e (%) = | 309 (5) [M⁺ + 2], 308 (35) [M⁺ + 1], 307 (91) | |
| | [M⁺], 264 100), 248 (43), 220 (41), 192 (6), | |
| | 166 (3), 145 (2), 129 (16), 118 (31), 118 | |
| | (31), 102 (23), 91 (3) | |
| ¹H—NMR (300 MHz, d₆-DMSO): | | |
| δ = | 8.40 | (s, 1H, C-3) |
| | 8.03 | (m, 1H, aryl, arom.) |
| | 7.99 | (m, 1H, aryl, arom.) |
| | 7.80-7.89 | (m, 4H, hetaryl, arom.) |
| | 7.58 | (m, 1H, aryl arom.) |
| | 7.48 | (m, 1H, aryl arom.) |
| | 5.18 | (sep, 1H, CH) |
| | 1.32 | (s, 3H, CH₃) |
| | 1.24 | (s, 3H, CH₃) |

### Example B

Preparation of N-(2-quinoxalinyl)-2-aminopyrimidine

1 mmol of 2-chloroquinoxaline and 1.5 mmol of 2-aminopyrimidine are dissolved in an inert solvent and refluxed for 4 hours. The solid which precipitates during the reaction is, after cooling, separated off by filtration and purified by recrystallisation, giving N-(2-quinoxalinyl)-2-aminopyrimidine.

Characterisation:

IR (KBr):

| | | |
|---|---|---|
| Wavenumber | 3427 | (ν(N—H), NH) |
| [cm − 1] = | | |
| | 3021 | (ν(C—H), Ph) |
| | 1597 | (ν(C═C), Ph) |
| | 1493 | (ν(C═C), Ph) |
| | 1262 | ((skeleton), Ph) |
| | 814 | (γ (C—H), Ph) |
| | 788 | (γ(C—H), Ph) |

MS:

| | |
|---|---|
| m/e (%) = | 224 (97) [M⁺ + 1], 223 (100) [M⁺], 222 (12) [M⁺ − 1], |
| | 195 (6), 170 (7), 143 (4) 129 (9), 102 (16), 90 |
| | (11), 79 (18) |

¹H—NMR (300 MHz, d₆-DMSO):

| | | |
|---|---|---|
| δ = | 10.70 | (br. s, 1H, NH) |
| | 9.71 | (s, 1H, hetaryl, arom.) |
| | 8.64 | (d, 2H, hetaryl, arom.) |
| | 7.66-8.03 | (m, 4H, hetaryl, arom.) |

### Example C

### PREPARATION EXAMPLES

The following quinoxaline derivatives are obtained analogously to Example B from the corresponding starting materials:

a) from 2-chloro-2-phenylquinoxaline and 4-aminobenzoic acid, N-[2-(3-phenyl)quinoxalinyl]-4-aminobenzoic acid;

Characterisation

| IR (KBr): | | |
|---|---|---|
| Wavenumber | 3421 | (ν(N—H), NH) |
| [cm − 1] = | | |
| | 3085 | (ν(C—H), Ph) |
| | 1709 | (ν(C═O)) |
| | 1618 | (ν(C═C), Ph) |
| | 1526 | (ν(C═C), Ph) |
| | 1256 | ((skeleton), Ph) |
| | 814 | (γ(C—H), Ph) |
| | 742 | (γ(C—H), Ph) |
| MS: | | |
| m/e (%) = | 342 (16) [M⁺ + 2], 341 (75) [M⁺1], 340 (100) | |
| | [M⁺], 296 (18), 264 (2), 220 (11), 194 (17), | |
| | 166 (5), 137 (13), 102 (17), 90 (23), 77 (21) | |
| ¹H—NMR (300 MHz, d₆-DMSO): | | |
| δ = | 7.80-7.94 | (m, 4H, hetaryl, arom.) |
| | 7.51-7.65 | (m, 5H, aryl, arom.) |
| | 7.43 | (br. s, 1H, OH) |
| | 7.23 | (m, 4H, aryl, arom.) |

b) from 2-chloroquinoxaline and 2,4,6-trifluoroaniline, N-[bis(2-quinoxalinyl)]-2,4,6-trifluoroaniline;

Characterisation:

IR (KBr):

| | | |
|---|---|---|
| Wavenumber | 2925 | (ν(Cₛₚ^{₂}—H), Ph), |
| [cm⁻¹] = | | |
| | 1535 | (ν (C═C), Ph), |
| | 1122 | (ν (C—F)), |
| | 758 | (γ (C—H), Ph). |

¹H—NMR (300 MHz, d₆-DMSO):

| | | |
|---|---|---|
| δ = | 11.48 | (s, 1 H), |
| | 10.32 | (s, 1 H, arom.), |
| | 8.13-8.20 | (m, 1 H, arom.), |
| | 8.07-8.13 | (m, 1 H, arom.), |
| | 8.00-8.13 | (dd, 1 H, arom., ³J = 8.1 Hz, ⁴J = 0.8 Hz), |
| | 7.76-7.83 | (m, 2 H, arom.), |
| | 7.64 | (dd, 1 H, 8-H, ³J = 8.3 Hz, ⁴J = 1.4 Hz), |
| | 7.59 | (dt, 1 H, 7-H, ³J = 8.4 Hz, ⁴J = 1.4 Hz), |
| | 7.46 | (dt, 1 H, 6-H, ³J = 6.6 Hz, ⁴J = 1.7 Hz), |
| | 7.19 | (s, 3 H, arom.), |
| | 6.75-6.85 | (m, 2 H, arom.). |

MS:

| | |
|---|---|
| m/e (%) = | 405 (2) [M⁺ + 2], 404 (16) [M⁺ + 1], |
| | 403 (64) [M⁺], 384 (100), 357 (5), 281 (6), |
| | 256 (6), 202 (12), 144 (27), 129 (12), 102 (17), 90 (13), |
| | 77 (11), 57 (6). |

c) from 2-chloroquinoxaline and 2-amino-4,5-dimethoxybenzoic acid, N-(2-quinoxalinyl)-2-amino-4,5-dimethoxybenzoic acid;

Characterisation:
Melting point: 248° C.

IR (KBr):

| | | |
|---|---|---|
| Wavenumber | 3115 | (ν (N—H), NH), |
| [cm⁻¹] = | | |
| | 2991 | (ν (Cₛₚ^{₂}—H), Ph), |
| | 1743 | (ν (C═O), CO₂H), |
| | 1637 | (ν (C═C), Ph), |
| | 1533 | (ν (C═C), Ph), |
| | 1246 | (δ (C—O—H), CO₂H), |
| | 753 | (γ (C—H), Ph). |

¹H-NMR (300 MHz, d₆-DMSO):

| | | |
|---|---|---|
| δ = | 11.42 | (s, 1 H, NH), |
| | 9.06 | (s, 1 H, 6′-H), |
| | 8.52 | (s, 1 H, 3-H), |
| | 7.88 | (dd, 1 H, 5-H, ³J = 8.1 Hz, |
| | | ⁴J = 0.9 Hz), |
| | 7.76 | (dd, 1 H, 8-H, ³J = 8.2 Hz, |
| | | ⁴J = 1.0 Hz), |
| | 7.67 | (dt, 1 H, 7-H, ³J = 7.6 Hz, |
| | | ⁴J = 1.3 Hz), |
| | 7.51 | (dt, 1 H, 6-H, ³J = 7.5 Hz, |
| | | ⁴J = 1.4 Hz), |
| | 7.46 | (s, 1 H, 3′-H), |
| | 3.95 | (s, 3 H, OCH₃), |
| | 3.77 | (s, 3 H, OCH₃). |

¹³C—NMR (75.4 MHz, d₆-DMSO):

| | | |
|---|---|---|
| δ = | 169.62 | (CO₂H), |
| | 153.31 | (C-4′), |
| | 148.41 | (C-2), |
| | 142.38 | (C-5′), |
| | 141.13 | (C-3), |
| | 139.83 | (C-8a), |
| | 138.53 | (C-2′), |
| | 137.04 | (C-4a), |
| | 130.08 | (C-7), |
| | 128.34 | (C-5), |
| | 126.76 | (C-6), |
| | 125.55 | (C-8), |
| | 112.98 | (C-6′), |
| | 106.28 | (C-1′), |
| | 102.66 | (C-3′), |
| | 55.51 | (OCH₃). |

MS:

| | |
|---|---|
| m/e (%) = | 327 (2) [M⁺ + 2], 326 (19) [M⁺ + 1], 325 (100) |
| | [M⁺], 307 (30), 292 (25), 280 (83), 266 (45), 236 (13), |
| | 221 (5), 206 (10), 193 (27), 179 (3), 145 (3), 129 (50), 102 |
| | (36), 90 (7), 76 (8), 64 (4). |

d) from 2-chloroquinoxaline and 4-aminomethylbenzoic acid, N-(2-quinoxalinyl)-4-aminomethylbenzoic acid;

e) from 2-chloroquinoxaline and 3,4,5-trimethoxyaniline, N-(2-quinoxalinyl)-3,4,5-trimethoxyaniline;

f) from 2-chloroquinoxaline and 2-aminoanisole, N-(2-quinoxalinyl)-2-aminoanisole;

Characterisation:
Melting point: 216° C.

IR (KBr):

| | | |
|---|---|---|
| Wavenumber | 3047 | (ν (Cₛₚ^{₂}—H), Ph), |
| [cm⁻¹] = | | |
| | 2784 | (ν (N—H), NH), |
| | 1656 | (ν (C═C), Ph), |
| | 1509 | (ν (C═C), Ph), |
| | 1251 | (ν (C═O), R—O—R), |
| | 835 | (γ (C—H), Ph), |
| | 756 | (γ (C—H), Ph). |

¹H—NMR (300 MHz, d₆-DMSO):

| | | |
|---|---|---|
| δ = | 10.07 | (s, 1 H, NH), |
| | 8.56 | (s, 1 H, 3-H), |
| | 7.88 | (d, 2 H, 2′-H, 6′-H, ³J = 9.0 Hz), |
| | 7.84 | (dd, 1 H, 5-H, ³J = 8.4 Hz, ⁴J = 0.7 Hz), |
| | 7.68 | (dd, 1 H, 8-H, ³J = 8.3 Hz, ⁴J = 1.4 Hz), |
| | 7.63 | (dt, 1 H, 7-H, ³J = 8.2 Hz, ⁴J = 1.4 Hz), |
| | 7.44 | (dt, 1 H, 6-H, ³J = 7.4 Hz, ⁴J = 1.6 Hz), |
| | 6.99 | (d, 2 H, 3′-H, 5′-H, ³J = 9.0 Hz), |
| | 3.77 | (s, 3 H, OCH₃). |

¹³C NMR (75.4 MHz, d₆-DMSO):

| | | |
|---|---|---|
| δ = | 154.71 | (C-4′), |
| | 149.42 | (C-2), |
| | 140.75 | (C-3), |
| | 140.15 | (C-8a), |
| | 136.47 | (C-4a), |
| | 133.09 | (C-1′), |
| | 129.99 | (C-7), |
| | 128.36 | (C-5), |
| | 125.66 | (C-6), |
| | 124.52 | (C-8), |
| | 120.45 | (C-2′, C-6′), |
| | 114.03 | (C-3′, C-5′), |
| | 55.15 | (OCH₃). |

MS:

| | |
|---|---|
| m/e (%) = | 253 (2) [M⁺ + 2], 252 (21) [M⁺ + 1], 251 (100) [M⁺], 236 |
| | (87), 220 (2), 208 (19), 181 (4), 154 (1), 129 (44), 118 |
| | (8), 102 (39), 90 (12), 76 (15), 63 (14). |

g) from 2-chloroquinoxaline and 2-aminobenzophenone, N-(2-quinoxalinyl)-2-aminobenzophenone.

Characterisation:
Melting point: 158° C.

IR (KBr):

| | | |
|---|---|---|
| Wavenumber | 3274 | (ν (N—H), NH), |
| [cm⁻¹] = | | |
| | 3123 | (ν (Cₛₚ^{₂}—H), Ph), |
| | 1603 | (ν (C═C), Ph), |
| | 1534 | (ν (C═C), Ph), |
| | 1265 | (skeleton Ph-CO—), |
| | 755 | (γ (C—H), Ph), |
| | 698 | (γ (C—H), Ph). |

¹H—NMR (300 MHz, d₆-DMSO):

| | | |
|---|---|---|
| δ = | 10.16 | (s, 1 H, NH), |
| | 8.40 | (s, 1 H, 3-H), |
| | 7.88 | (d, 1 H, 5-H, ³J = 7.9 Hz), |
| | 7.76 | (dd, 1 H, 8-H, ³J = 7.9 Hz, ⁴J = 1.6 Hz), |
| | 7.73 | (dd, 1 H, arom., ³J = 8.4 Hz, ⁴J = 1.4 |
| | | Hz), |
| | 7.58-7.70 | (m, 2 H, arom.), |
| | 7.36-7.50 | (m, 5 H, arom.), |
| | 7.27 | (dt, 1 H, arom., ³J = 7.6 Hz, ⁴J = 1.3 Hz), |
| | 7.06 | (dd, 1 H, arom., ³J = 8.1 Hz, ⁴J = 1.4 |
| | | Hz). |

¹³C—NMR (75.4 MHz, d₆-DMSO):

| | | |
|---|---|---|
| δ = | 157.65 | (C-1′), |
| | 148.98 | (C-2), |
| | 139.86 | (C-3), |
| | 137.76 | (C-8a), |
| | 137.13 | (C-4a), |
| | 132.53 | (C ₜₑᵣₜ arom.), |
| | 132.04 | (C-4″), |
| | 130.40 | (C ₜₑᵣₜ arom.), |
| | 129.76 | (C-7), |
| | 129.57 | (C-2″, C-6″), |
| | 128.30 | (C-5), |
| | 128.10 | (C-3″, C-5″), |
| | 125.40 | (C-6), |
| | 124.92 | (C-8), |
| | 122.90 | (C ₜₑᵣₜ arom.), |
| | 122.63 | (C ₜₑᵣₜ arom.). |

MS:

| | |
|---|---|
| m/e (%) = | 327 (1) [M+ + 2], 326 (10) [M+ + 1], 325 (41) [M+], 296 |
| | (32), 248 (10), 220 (100), 196 (5), 152 (5), 118 (5), |
| | 105 (12), 92 (10), 77 (34), 65 (5). |

h) from 2-chloro-3 phenylquinoxaline and 2-amino-4,5-dimethoxybenzoic acid,

2-(3-phenylquinoxalin-2-ylamino)-4,5-dimethoxybenzoic acid

Characterisation:
Melting point: 126° C.

IR (KBr):

| | | |
|---|---|---|
| Wavenumber | 3422 | (ν (N—H), NH), |
| [cm⁻¹] = | | |
| | 1995 | (ν (Cₛₚ_{₂}—H), Ph), |
| | 2828 | (ν (Cₛₚ_{₃}—H), CH₃), |
| | 1604 | (ν (C═O), CO₂H), |
| | 1537 | (ν (C═C), Ph), |
| | 1228 | (ν (C—O), R—O—R), |
| | 863 | (γ (C—H), Ph), |
| | 753 | (γ (C—H), Ph). |

¹H—NMR (300
MHz, d₆-DMSO):

| | | |
|---|---|---|
| δ = | 8.31 | (s, 1 H, NH), |
| | 7.83-7.90 | (m, 3 H, arom.), |
| | 7.66 | (dt, 1 H, arom., ³J = 7.2 Hz, |
| | | ⁴J = 1.0 Hz), |
| | 7.55-7.66 | (m, 5 H, arom.), |
| | 7.47 | (dt, 1 H, 6-H, ³J = 7.4 Hz, |
| | | ⁴J = 1.5 Hz), |
| | 7.38 | (dt, 1 H, arom., ³J = 8.7 Hz, |
| | | ⁴J = 2.9 Hz), |
| | 6.93 | (d, 1 H, arom., ³J = 8.8 Hz), |
| | 3.77 | (s, 3 H, OCH₃), |
| | 3.76 | (s, 3 H, OCH₃). |

¹³C—NMR (75.4
MHz, d₆-DMSO):

| | | |
|---|---|---|
| δ = | 148.33 | (C-2) |
| | 147.66, | (C _{quat.} arom.), |
| | 147.20, | |
| | 144.53 | |
| | 140.01 | (C-1″), |
| | 136.98 | (C-8a), |
| | 136.65 | (C-4a), |
| | 133.36 | (C-2′), |
| | 129.77 | (C-7), |
| | 129.52 | (C-3″, C-5″), |
| | 128.72 | (C-2″, C-4″, C-6″), |
| | 128.27 | (C-5), |
| | 125.91 | (C-6), |
| | 125.04 | (C-8), |
| | 112.93, | (C _{quat.} arom.), |
| | 111.9, | |
| | 106.3 | |
| | 55.77 | (OCH₃), |
| | 55.35 | (OCH₃). |

MS (EI):

| | |
|---|---|
| m/e (%) = | 391 [M⁺] (2), 359 (39), 357 (100), 342 (61), |
| | 327 (1), 299 (4), 270 (6), 220 (2), 205 (25), 179 |
| | (10), 150 (3), 102 (10), 90 (4), 77 (17), 65 (2). |

MS (FD):

| | |
|---|---|
| m/e (%) = | 401 [M⁺] (2), 391 (6), 357 (100), 178 (2), 78 (4). |

### Example D

Preparation of 5-nitro-2,3,6-trimethoxyquinoxaline (A) and 2,3,7-trimethoxy-6,8-dinitroquinoxaline (B)

A mixture of 3.77 mmol of conc. nitric acid and 10 ml of conc. sulfuric acid is added at a temperature of 0° C. to a solution of 3.77 mmol of 2,3,6-trimethoxyquinoxaline in conc. sulfuric acid (10 ml). The reaction mixture is stirred at 0° C. for 30 minutes and poured onto ice (100 g). 8 N sodium hydroxide solution is added until the mixture is neutral, and the mixture is extracted with dichloromethane (3×50 ml). The combined organic phases are dried over magnesium sulfate, and the solvent is distilled off. The product mixture is separated by column chromatography on silica gel using cyclohexane/ethyl acetate (4:1). The separated products are recrystallised from methanol. Substance (A) is obtained in the form of yellow crystals in a yield of 72%, and compound (B) is obtained as yellow crystals in a yield of 4%.

Characterisation:

2,3,6-trimethoxy-5,7-dinitroquinoxaline:
Beige crystals
Melting point: 143-144° C.
300 MHz-¹H—NMR (CDCl₃):

| | | |
|---|---|---|
| δ = | 8.40 | (s, 1H, H-8) |
| | 4.08 | (s, 3H, —OCH₃) |
| | 4.06 | (s, 3H, —OCH₃) |
| | 4.02 | (s, 3H, —OCH₃) |

75 MHz-¹³C—NMR (CDCl₃):

| | | |
|---|---|---|
| δ = | 153.31 | (quat. C) |
| | 151.76 | (quat. C) |
| | 143.62 | (quat. C) |
| | 141.65 | (quat. C) |
| | 141.17 | (quat. C) |
| | 132.98 | (quat. C) |
| | 132.83 | (quat. C) |
| | 124.96 | (tert C) |
| | 65.09 | (—OCH₃) |
| | 55.80 | (—OCH₃) |
| | 55.41 | (—OCH₃) |

MS

| | |
|---|---|
| m/e (%) = | 300 (100) [M⁺], 219 (16), 176 (10), 89 (10) |

C₁₁H₁₀N₄O₇ (310.22 g/mol)

| | | | |
|---|---|---|---|
| Calc. [%] | C 42.59 | H 3.25 | N 18.06 |
| Found [%] | C 42.58 | H 3.15 | N 18.21 |

2,3,6-trimethoxy-5-nitroquinoxaline:
Yellow crystals
Melting point: 185-186° C.
300 MHz-¹H—NMR (CDCl₃):

| | | |
|---|---|---|
| δ = | 7.73 | (d, 1H, ³J = 9.2 Hz, H-8) |
| | 7.18 | (d, 1H, ³J = 9.2 Hz, H-7) |
| | 4.04 | (s, 3H, —OCH₃) |
| | 4.02 | (s, 3H, —OCH₃) |
| | 3.96 | (s, 3H, —OCH₃) |

75 MHz-¹³C—NMR (CDCl₃):

| | | |
|---|---|---|
| δ = | 151.69 | (quat. C) |
| | 149.56 | (quat. C) |
| | 148.90 | (quat. C) |
| | 135.03 | (quat. C) |
| | 131.37 | (quat. C) |
| | 130.08 | (quat. C) |
| | 128.65 | (tert C) |
| | 111.93 | (tert C) |
| | 57.03 | (—OCH₃) |
| | 55.03 | (—OCH₃) |
| | 54.64 | (—OCH₃) |

MS(70 eV):

| | |
|---|---|
| m/e (%) = | 266 (13) [M⁺ + 1], 265 (100) [M⁺], 231 (13), 189 (19) |

C₁₁H₁₁N3O5 (265.23 g/mol)

| | | | |
|---|---|---|---|
| Calc. [%] | C 49.81 | H 4.18 | N 15.84 |
| Found [%] | C 49.66 | H 4.21 | N 15.92 |

### Example E

Preparation of N,N-benzoyl-(2-quinoxalinyl)-4-aminobenzoic Acid

A mixture of 2 mmol of benzoic anhydride and 2 mmol of trimethylsilyl N-(2-quinoxalinyl)-4-aminobenzoate [which can be prepared by reaction of N-(2-quinoxalinyl)-4-aminobenzoic acid with trimethylsilyl cyanide in acetonitrile under a nitrogen atmosphere] in 7 ml of dichloromethane is added in portions under a nitrogen atmosphere to a suspension of 0.55 mmol of AgOTf (silver triflate) and 0.275 mmol of TiCl₄ (titanium tetrachloride) in 5 ml of dichloromethane. A solution of 2 mmol of 2-propyl trimethylsilyl ether (which can be prepared by reaction of trimethylchlorosilane with N,N-dimethylaniline in a solution of propan-2-ol) is subsequently added dropwise, and the reaction mixture is stirred at room temperature for 20 hours. After aqueous saturated NaHCO₃ solution has been added, the mixture is extracted with chloroform, and, after separation, the organic phase is dried over sodium sulfate. Further purification is carried out by column chromatography on silica gel using cyclohexane/ethyl acetate (1:1). N,N-Benzoyl-(2-quinoxalinyl)-4-aminobenzoic acid can be obtained as a pale-yellow powder in a yield of 27%.

Characterisation:

IR (KBr):

| | | |
|---|---|---|
| Wavenumber | 3354 | (ν(—NR₂) |
| [cm − 1] = | | |
| | 3123 | (ν(C—H), Ph) |
| | 1789 | (ν(O═C—NR₂) |
| | 1712 | (ν(C═O)) |
| | 1600 | (ν(C═C), Ph) |
| | 1521 | (ν(C═C), Ph) |
| | 1228 | (ν(skeleton), Ph-C—O—) |
| | 758 | (γ(C—H), Ph) |
| | 708 | (γ(C—H), Ph) |

MS:

| | |
|---|---|
| m/e (%) = | 371 (10) [M⁺ + 2], 370 (32) [M⁺ + 1], 369 (100) |
| | [M⁺], 345 (8), 264 (6), 220 (7), 122 (8), 105 |
| | (4), 78 (9) |

¹H—NMR (300 MHz, d₆-DMSO):

| | | |
|---|---|---|
| δ = | 8.64 | (s, 1H, 3-H) |
| | 7.49-7.68 | (m, 5H, benzoyl, |
| | | arom.) |
| | 7.82-7.97 | (m, 4H, aryl, arom.) |
| | 8.09-8.26 | (m, 4H, hetaryl, |
| | | aromat.) |

### Example F

Preparation of diethyl N-(2-quinoxalinyl)]-4-aminobenzoylglutamate

A mixture of 1.2 mmol of N-(2-quinoxalinyl)-4-aminobenzoic acid and 1.3 mmol of diethyl-L-glutamate hydrochloride in 24 ml of dry DMF are added at room temperature and under a nitrogen atmosphere to a solution of 1.3 mmol of diethyl cyanophosphonate in 2.5 mmol of triethylamine. After stirring for 1.5 hours, the solution is taken up in a mixture of ethyl acetate/benzene (ratio 3:1) and washed with water and aqueous saturated NaHCO₃ solution. The organic phase is separated off, dried over sodium sulfate and purified by column chromatography on silica gel using cyclohexane/ethyl acetate (1:2.5), giving diethyl N-(2-quinoxalinyl)]-4-aminobenzoylglutamate in a yield of 71%.

Characterisation:

IR (KBr):

| | | |
|---|---|---|
| Wavenumber | 3383 | (ν(N—H), NH) |
| [cm − 1] = | | |
| | 3312 | (ν(N—H), NH) |
| | 2982 | (ν(C—H), alkyl) |
| | 1747 | (ν(C═O) |
| | 1642 | (ν(C═C),Ph) |
| | 1544 | (ν(C═C),Ph) |
| | 1270 | ((skeleton), Ph) |
| | 856 | (γ(C—H), Ph) |
| | 765 | (γ(C—H), Ph) |

MS:

| | |
|---|---|
| m/e (%) = | 451 (4) [M⁺ + 1], 450 (14) [M⁺], 404 (9), 377 (2), |
| | 322 (3), 293 (15), 264 (12), 248 (100), 220 |
| | (26), 202 (10), 151 (3), 120 (42), 84 (35) |

¹H—NMR (300 MHz, d₆-DMSO):

| | | |
|---|---|---|
| δ = | 8.48 | (s, 1H, H-3) |
| | 7.84-7.96 | (m, 4H, hetaryl, arom.) |
| | 7.03-7.69 | (m, 2H, aryl, arom.) |
| | 7.48-7.54 | (m, 2H, aryl, arom.) |
| | 4.80 | (superimposed + 1H, CH) |
| | 4.24 | (dt, 2H, CH₂) |
| | 4.12 | (dt, 2H, CH₂) |
| | 2.46 | (q, 2H, CH₂CH₃) |
| | 2.16 | (q, 2H, CH₂CH₃) |
| | 1.28 | (t, 3H, CH₃) |
| | 1.20 | (t, 3H, CH₃) |

### Example G

### 2-chloro-3-(2-triisopropylsilylacetylenyl)quinoxaline

Preparation: 10 mol % of a mixture of Pd catalyst (palladium (Pd))/triarylphosphine/CuI (copper iodide) in a ratio of 1:2:0.75 in 1.5 ml of CH₃CN are added to a solution of 1.00 mmol of dichloroquinoxaline 4 ml of CH₃CN (acetonitrile). 12 mmol of Et₃N (triethylamine) and 1.20 mmol of triisopropylsilylacetylene are subsequently added. After a reaction time of 2 hours under a nitrogen atmosphere at room temperature, the solvent is removed, and the mixture is quenched with 3 ml of H₂O. The aqueous phase is extracted with dichloromethane, and the organic phase is separated off and dried over magnesium sulfate. Purification is carried out by column chromatography on silica gel. 2-Chloro-3-(2-triisopropylsilylacetylenyl)quinoxaline is obtained as a pale-yellow oil in a yield of 79%.

Characterisation:

IR (KBr):

| | | |
|---|---|---|
| Wavenumber | 3072 | (ν(C—H), Ph) |
| [cm − 1] = | | |
| | 2947 | (ν(C—H), alkyl) |
| | 2071 | (ν(C═H), alkynyl) |
| | 1611 | (ν(C═C), Ph) |
| | 1558 | (ν(C═C), Ph) |
| | 1314 | ((skeleton), Ph) |
| | 1288 | ((skeleton), Ph) |
| | 762 | (γ(C—H), Ph) |
| | 683 | (γ(C—H), Ph) |

MS:

| | |
|---|---|
| m/e (%) = | 346 (11) [M⁺ + 2], 344 (33) [M⁺], 303 (44, 301 (100), |
| | 273 (10), 261 (29), 259 (70), 233 (2.7), 231 (6), 195 |
| | (15), 181 (11), 163 (1) 153 (7), 129 (9), 117 (13), 103 |
| | (35), 93 (14), 79 (5) |

¹H—NMR (300 MHz, d-CDCl₃):

| | | |
|---|---|---|
| δ = | 7.98 | (m, 1H, hetaryl, |
| | | arom.) |
| | 7.88 | (m, 1H, hetaryl, |
| | | arom.) |
| | 7.67 | (m, 2H, hetaryl, |
| | | arom.) |
| | 1.33 | (s, 3H, C—H) |
| | 1.09 | (s, 21, CH₃) |

¹³C (75.4 MHz)

| | | |
|---|---|---|
| δ = | 148.46 | |
| | 140.82 | |
| | 140.51 | |
| | 135.49 | |
| | 130.77 | (C_{quat} arom.) |
| | 129.11 | (C_{quat} arom.) |
| | 128.37 | (C_{quat} arom.) |
| | 127.83 | |
| | 102.22 | (C-1; alkynyl) |
| | 98.97 | (C-1; alkynyl) |
| | 18.46 | (CH₃) |
| | 11.64 | (C—H) |

The following was prepared analogously: 2-amino-3-(2-phenylacetylenyl)-quinoxaline

Characterisation:

IR (KBr):

| | | |
|---|---|---|
| Wavenumber | 3466 | |
| [cm − 1] = | | |
| | 3316 | (ν(N—H), NH) |
| | 3058 | (ν(C—H), Ph) |
| | 2203 | (ν(C═H), alkynyl) |
| | 1631 | (ν(C═C), Ph) |
| | 1558 | (ν(C═C), Ph) |
| | 1295 | ((skeleton), Ph) |
| | 755 | (γ(C—H), Ph) |
| | 690 | (γ(C—H), Ph) |

MS:

| | |
|---|---|
| m/e (%) = | 246 (18) [M⁺ + 1], 245 (100) [M⁺], 218 (12), 190 (1), |
| | 142 (11), 127 (6), 102 (6), 91 (19), 76 (9) |

¹H—NMR (300 MHz, d-CDCl₃):

| | | |
|---|---|---|
| δ = | 7.78-7.86 | (m, 3H, hetaryl, |
| | | arom.) |
| | 7.49-7.60 | (m, 5H, arom.) |
| | 7.40-7.42 | (m, 1H, hetaryl, |
| | | arom.) |
| | 7.11 | (br. s, 1H, NH₂) |
| ¹³C (75.4 MHz) | 152.92 | (C_{quat.} arom.) |
| | 140.98 | (C_{quat.} arom.) |
| | 136.41 | (C_{quat.} arom.) |
| | 132.34 | (C_{quat.} arom.) |
| | 130.54 | (C_{quat.} arom.) |
| | 129.60 | (C_{quat.} arom.) |
| | 129.95 | (C—H arom.) |
| | 128.66 | (C—H arom.) |
| | 128.10 | (C—H arom.) |
| | 125.19 | (C—H arom.) |
| | 124.34 | (C—H arom.) |
| | 120.85 | (C_{quat.} arom.) |
| | 94.80 | (C-1, alkynyl) |
| | 85.011 | (C-2, alkynyl) |

### Example H

### 3-[2-(p-tolyl)ethenyl]quinoxalin-2-one

Preparation: 1.05 mmol of 4-methylbenzylidenebenzotartaric acid and 1.00 mmol of phenylenediamine are warmed to a temperature of 40° C. in a mixture of 6 ml of ethanol (absolute) and 6 ml of glacial acetic acid. After reaction times of 0.5-1.5 hours, a microcrystalline solid precipitates. After recrystallisation from ethanol, the pale-yellow product can be obtained in yields of 74-86%.

Characterisation:

IR (KBr):

| | | |
|---|---|---|
| Wavenumber | 3421 | (ν(N—H), NH) |
| [cm − 1] = | | |
| | 3033 | (ν(C—H), Ph) |
| | 1670 | (ν(C═O), amide) |
| | 1611 | (ν(C═N), hetaryl) |
| | 1525 | (ν(C═C), Ph) |
| | 1328 | ((skeleton), Ph) |
| | 768 | (γ(C—H), Ph) |

MS:

| | |
|---|---|
| m/e (%) = | 262 (22) [M⁺], 247 (14), 233 (100), 218 (12), 208 |
| | (33), 190 (9), 145 (59), 131 (5), 115 (49), 102 (9), 91 |
| | (31), 77 (9) |

¹H—NMR (300 MHz, d-CDCl₃): xx

| | | |
|---|---|---|
| δ = | 12.6 | (br.s, 1H, N—H) |
| | 8.14-8.19 | (m, 1H, CH) |
| | 7.89 | (m, 1H, CH) |
| | 7.80 | (m, 2H, hetaryl) |
| | 7.67-7.76 | (m, 2H, hetaryl) |
| | 7.32-7.46 | (m, 4H, aryl) |

### Example I

### N-2-[3-(ethoxycarbonyl)quinoxalinyl]-4-aminobenzoic Acid

Preparation: 2-chloro-3-ethoxycarbonyl ester quinoxaline (100 g, 4.46 mmol) and p-aminobenzoic acid (0.91 g, 6.69 mmol) are refluxed in 20 ml of EtOH. The product precipitates as a yellow solid during the reaction. The product is filtered off with suction and recrystallised from EtOH, giving N-2[3-(ethoxycarbonyl)quinoxalinyl]-4-aminobenzoic acid as a yellow solid.

Characterisation:

| IR (KBr): | | |
|---|---|---|
| Wavenumber | 3480 | (ν(O—H), OH) |
| [cm − 1] = | | |
| | 3270 | (ν(N—H), NH) |
| | 3012 | (ν(C—H), Ph) |
| | 2976 | (ν(C—H), ethyl) |
| | 1703 | (ν(C═O) CO₂Et) |
| | 1696 | (ν(C═O) CO₂H) |
| | 1613 | (ν(C═C), Ph) |
| | 1538 | (ν(C═C), Ph) |
| | 1328 | ((skeleton), Ph) |
| | 1292 | ((skeleton), Ph) |
| | 768 | (γ(C—H), Ph) |
| MS: | | |
| m/e (%) = | 338 (19) [M⁺ + 1], 337 (100) [M⁺], 336 (55), | |
| | 292 (5), 264 (17), 246 (6) 219 (76), 102 (4), | |
| | 90 (15), 77 (3) | |
| ¹H—NMR (300 MHz, d₆-DMSO): | | |
| δ = | 12.72, | (br. s, 1H, OH) |
| | 10.40 | (br. s, 1H, NH) |
| | 7.91-8.25 | (m, 5H, arom.) |
| | 7.81-7.85 | (m, 2H, hetaryl, arom.) |
| | 7.57-7.66 | (m, 2H, hetaryl, arom.) |
| | 4.49 | (q, 2H, CH₂) |
| | 1.43 | (t, 3H, CH₃) |

The following was prepared analogously: N-2-[3-(ethoxycarbonyl)-quinoxalinyl]-4-acetylaniline

IR (KBr):

| | | |
|---|---|---|
| Wavenumber | 3269 | (ν(N—H), NH) |
| [cm − 1] = | | |
| | 2987 | (ν(C—H), ethyl) |
| | 1703 | (ν(C═O) CO₂Et) |
| | 1676 | (ν(C═O) COCH₃) |
| | 1664 | (ν(C═C), Ph) |
| | 1538 | (ν(C═C), Ph) |
| | 1275 | ((skeleton), Ph) |
| | 769 | (γ(C—H), Ph) |

MS:

| | |
|---|---|
| m/e (%) = | 335 (38) [M⁺], 320 (6), 252 (55), 246 (12), 237 |
| | (100), 219 (29) , 194 (2), 155 (3), 135 (19), 120 |
| | (29), 92 (20), 77 (5) |

¹H—NMR (300 MHz, d₆-DMSO):

| | | |
|---|---|---|
| δ = | 10.41 | (br. s, 1H, NH) |
| | 7.99-8.11 | (m, 5H, arom.) |
| | 7.81-7.85 | (m, 2H, hetaryl, arom.) |
| | 7.57-7.66 | (m, 2H, hetaryl, arom.) |
| | 4.49 | (q, 2H, CH₂) |
| | 2.56 | (s, 3H, COCH₃) |
| | 1.43 | (t, 3H, CH₃) |

### Example K

### 1-(2-quinoxalinyl)-3-[4-(iso-propyl)phenyl]prop-2-en-1-one (A) and 1-(2-quinoxalinyl)-3-[3,4-(oxolano)phenyl]prop-2-en-1-one (B)

1.00 mmol of 2-acetylquinoxaline is dissolved in 3 ml of ethanol (anhydrous), and 1.05 mmol of the aldehyde are added with stirring at a temperature of −10-0° C. 0.40 mmol of base (ethanolic KOH or piperidine) is subsequently added dropwise at this temperature. After the solution has been stirred for a further 1 hour at a temperature of 0° C., it is allowed to warm slowly to room temperature and to react for 2-6 hours. During the reaction time, a solid precipitates, which can be purified, after filtration of the solution, by recrystallisation using ethanol/ethyl acetate. The compounds can be obtained in yields of 46-73%.

Characterisation A

IR (KBr):

| | | |
|---|---|---|
| Wavenumber | 3063 | (ν(C—H), Ph) |
| [cm − 1] = | | |
| | 2983 | (ν(C—H), alkyl) |
| | 1670 | (ν(C═O)) |
| | 1611 | (ν(C═C), Ph) |
| | 1349 | ((skeleton), Ph) |
| | 788 | (γ(C—H), Ph) |
| | 769 | (γ(C—H), Ph) |

MS:

| | |
|---|---|
| m/e (%) = | 301 (100) [M⁺], 286 (10), 272 (43), 258 (27), 243 (3), |
| | 230 (55), 203 (1), 172 (17), 155 (4), 130 (84), 114 |
| | (5), 103 (7), 91 (2), 77 (2) |

¹H—NMR (300 MHz, CDCL₃):

| | | |
|---|---|---|
| δ = | 9.56 | (s, 1H, H-3) |
| | 8.22 | (d, 1H, HC═C) |
| | 8.17-8.20 | (m, 1H, hetaryl, arom.) |
| | 8.10-8.13 | (m, 1H, hetaryl, arom.) |
| | 7.95 | (d, 1H, C═CH) |
| | 7.77-7.84 | (m, 2H, hetaryl, arom.) |
| | 7.62-7.65 | (m, 2H, arom.) |
| | 7.18-7.25 | (m, 2H, arom.) |
| | 2.88 | (sep., 1H, CH) |
| | 1.22 | (s, 3H, CH₃) |
| | 1.19 | (s, 3H, CH₃) |

Characterisation B

IR (KBr):

| | | |
|---|---|---|
| Wavenumber | 3065 | (ν(C—H), Ph) |
| [cm − 1] = | | |
| | 2933 | (ν(C—H), alkyl) |
| | 1670 | (ν(C═O)) |
| | 1591 | (ν(C═C), Ph) |
| | 1268 | ((skeleton), Ph) |
| | 1243 | ((skeleton), Ph) |
| | 813 | (γ(C—H), Ph) |
| | 782 | (γ(C—H), Ph) |

MS:

| | |
|---|---|
| m/e (%) = | 304 (100) [M⁺], 290 (4), 288 (2), 276 (9), 258 (37), |
| | 232 (12), 230 (47), 204 (15), 172 (6), 159 (3), 130 |
| | (71), 102 (17), 91 (11), 77 (2) |

¹H—NMR (300 MHz, CDCL₃):

| | | |
|---|---|---|
| δ = | 9.55 | (s, 1H, H-3) |
| | 8.17-8.20 | (m, 1H, hetaryl, arom.) |
| | 8.13 | (d, 1H, HC═C) |
| | 8.04-8.11 | (m, 1H, hetaryl, arom.) |
| | 7.90 | (d, 1H, C═CH) |
| | 7.77-7.85 | (m, 2H, hetaryl, arom.) |
| | 7.24-7.26 | (m, 1H, arom.) |
| | 7.15-7.19 | (m, 1H, arom.) |
| | 6.78-6.81 | (m, 1H, arom.) |
| | 6.27 | (s, 2H, CH₂) |

### Example L

N-[2-[N′-(benzamido)quinoxalinyl]-]benzamide

Preparation: 1.98 mmol of benzoyl chloride (freshly distilled) are added to a solution of 1.00 mmol of diaminoquinoxaline (160 mg) in 15.0 ml of a polar aprotic solvent (preferably THF or dioxane/dried), and the mixture is refluxed for 48-72 hours. After cooling, the solvent is removed, and the residue is purified by column chromatography on silica gel using a solvent/eluent mixture of cyclohexane/ethyl acetate→ethyl acetate/ethanol. The product can be obtained as a pale-yellow powder in a yield of 67%.

Characterisation:

IR (KBr):

| | | |
|---|---|---|
| Wavenumber | 3065 | (ν(C—H), Ph) |
| [cm⁻¹] = | | |
| | 1690 | (ν(C═O)) |
| | 1611 | (ν(C═C), Ph) |
| | 1532 | (ν(C═C), Ph) |
| | 1341 | ((skeleton), Ph) |
| | 1223 | ((skeleton), Ph) |
| | 775 | (γ(C—H), Ph) |
| | 709 | (γ(C—H), Ph) |

MS:

| | |
|---|---|
| m/e (%) = | 368 (10) [M⁺], 280 (4), 265 (55), 246 (100), 219 (5), |
| | 143 (55), 116 (23), 105 (10), 95 (7), 77 (8) |

¹H—NMR (300 MHz, d-CDCl₃):

| | | |
|---|---|---|
| δ = | 11.93 | (br.s, 1H, N—H) |
| | 8.43-8.46 | (m, 2H, hetaryl, arom.) |
| | 8.13-8.16 | (m, 2H, hetaryl, arom.) |
| | 7.94-7.98 | (m, 1H, aryl, arom.) |
| | 7.76-7.80 | (m, 2H, aryl, arom.) |
| | 7.65-7.70 | (m, 4H, aryl, arom.) |
| | 7.51-7.54 | (m, 1H, aryl, arom.) |
| | 7.08-7.16 | (m, 2H, aryl, arom.) |
| | 28.88 | (CH₃) |

### Example M

3-(ethyl)pyrido[1′,2′:1,2]imodazo[4,5-b]quinoxaline

Preparation: 3.00 mmol of pyridine are added to a solution of 1.00 mmol of 2-amino-3-chloroquinoxaline in 1.5 ml of DMF (dimethylformamide) or NMP (N-methylpyrrolidinone). The mixture is allowed to react at a temperature of 100° C. for 48-96 hours. After cooling, a small amount of H₂O is added to the solution. The precipitate which deposits can be purified by recrystallisation from a nonpolar solvent (using activated carbon).

Characterisation:

| IR (KBr): | | |
|---|---|---|
| Wavenumber | 3072 | (ν(C—H), Ph) |
| [cm − 1] = | | |
| | 2973 | (ν(C—H), ethyl) |
| | 1657 | (ν(C═N)) |
| | 1578 | (ν(C═C), Ph) |
| | 1523 | (ν(C═C), Ph) |
| | 1341 | ((skeleton), Ph) |
| | 1292 | ((skeleton), Ph) |
| | 755 | (γ(C—H), Ph) |
| | 742 | (γ(C—H), Ph) |
| MS: | | |
| m/e (%) = | 248 (100) [M⁺], 233 (24), 179 (18), | |
| | 144 (10), 117 (1), 90 (3), 77 (3) | |
| ¹H—NMR (300 MHz, d-CDCl₃): | | |
| δ = | 8.99-9.01 | (d, 1H, pyridyl) |
| | 8.20-8.26 | (m, 2H, hetaryl, arom.) |
| | 7.80-7.88 | (m, 2H, hetaryl, arom.) |
| | 7.59 | (s, 1H, pyridyl) |
| | 7.07-7.09 | (d, 1H, pyridyl) |
| | 2.83 | (q, 2H, CH₂) |
| | 1.31 | (t, 3H, CH₃) |

The following was prepared analogously:

3-(acetyl)pyrido[1′,2′:1,2]imodazo[4,5-b]quinoxaline

Characterisation:

IR (KBr):

| | | |
|---|---|---|
| Wavenumber | 3420 | (ν(C—N), NH) |
| [cm − 1] = | | |
| | 3072 | (ν(C—H), Ph) |
| | 1703 | (ν(C═O), COCH₃) |
| | 1643 | (ν(C═N)) |
| | 1630 | (ν(C═N)) |
| | 1583 | (ν(C═C), Ph) |
| | 1536 | (ν(C═C), Ph) |
| | 1367 | ((skeleton), Ph) |
| | 1281 | ((skeleton), Ph) |
| | 827 | (γ(C—H), Ph) |
| | 766 | (γ(C—H), Ph) |

MS:

| | |
|---|---|
| m/e (%) = | 262 (100) [M⁺], 247 (23), 234 (1), 219 (54), 192 (9), |
| | 164 (4), 129 (3), 102 (13), 90 (6), 76 (8) |

¹H—NMR (300 MHz, d-CDCl₃):

| | | |
|---|---|---|
| δ = | 9.05-9.10 | (d, 1H, pyridyl) |
| | 8.61 | (s, 1H, pyridyl) |
| | 8.40-8.43 | (m, 1H, hetaryl, arom.) |
| | 8.31-8.34 | (m, 1H, hetaryl, arom.) |
| | 7.89-7.97 | (m, 2H, hetaryl, arom.) |
| | 7.70 | (d, 1H, pyridyl) |
| | 2.85 | (s, 3H, CH₃) |

### Example N

carbohydrate-substituted quinoxaline derivatives was prepared as described in Morita, N., Inoue, K., Takagi, M., Agric. Biol. Chem. 1985, 49 (11), 3279-3289.

Preparation

A 10-fold molar excess of o-phenylenediamine is added to a solution of the corresponding mono- or disaccharide in MeOH/AcOH/H₂O (8:1:2), and the mixture is heated at a temperature of 80° C. for 3 hours. After cooling, the mixture is evaporated to dryness, and the residue is purified by column chromatography on silica gel (using chloroform/methanol 2:1). The glycosidic quinoxalines are obtained in yields of 21-35%.

Characterisation

2-[(1′R,2′S,3′R)-4′-(α-D-Glucopyranosyloxy)-1′,2′,3′-trihydroxybutyl]-quinoxaline

¹H NMR (300 MHz, CD₃OD):

δ 3.34 (dd, 1H, 4″-H), 3.43 (dd, 1H, 2″-H), 3.60-3.73 (m, 5H, 3″-H, 5″-H, 6″-Hₐ, 6″-H_{b}, 3′-H), 4.03-4.07 (m, 3H, 2′-H, 4′-H₂), 4.85 (d, 1H, 1″-H), 5.35 (s, 1H, 1′-H), 7.76-7.82 (m, 2H, 6-H, 7-H), 8.04-8.09 (m, 2H, 5-H, 8-H), 9.14 (s, 1H, 3-H).—J_{1″,2″}=J_{2″,3″}=9.6 Hz.

¹³C NMR (75.5 MHz, CD₃OD):

δ 62.5 (C-6″), 70.1 (C4′), 71.1 (C-3′), 71.6 (C-4″), 73.6 (C-1′), 73.7 (C-2″), 75.0 (C-5″), 75.2 (C-2′),100.2 (C-1″), 129.5, 129.8 (C-5, C-8), 130.8, 131.3 (C-6, C-7),142.5, 142.7 (C-4a, C-8a), 145.9 (C-3),159.7 (C-2).

MS (FD): m/e=435 [M+Na⁺].

6,7-Dichloro-2-[(1′R,2′S,3′R)-4′-(α-D-glucopyranosyloxy)-1′,2′,3′-trihydroxybutyl]quinoxaline

¹H NMR (300 MHz, CD₃OD):

δ 3.32 (dd, 1H, 4″-H), 3.45 (dd, 1H, 2″-H), 3.59-3.64 (m, 5H, 3″-H, 4″-H, 5″-H, 6″-Hₐ, 3′-H), 3.67-3.70 (m, 1H, 6″-H_{b}), 4.06-4.07 (m, 3H, 2′-H, 4′-H₂), 4.85 (s, 1H, 1″-H), 5.32 (s, 1H, 1′-H), 8.20, 8.21 (2 s, each 1H, 5-H, 8-H), 9.14 (s, 1H, 3-H).—J_{1″,2″}=3.5, J_{2″,3″}=9.6 Hz.

¹³C NMR (75.5 MHz, CD₃OD):

δ 63.0 (C-6″), 70.6 (C-4′), 71.5 (C-3′), 72.0 (C-4″), 74.0 (C-2″), 74.2 (C-1′, C-5″), 75.5 (C-3″), 75.7 (C-2′), 100.7 (C-1″), 131.0, 131.1 (C-5, C-8),135.1, 135.6 (C-6, C-7), 141.9 (C4a, C-8a), 147.8 (C-3), 161.8 (C-2).

2-[(1′R,2′S,3′R)-1′,2′,3′,4′-Tetrahydroxybutyl]quinoxaline-6-carboxylic Acid

and

2-[(1′R,2′S,3′R)-1′,2′,3′,4′-tetrahydroxybutyl]quinoxaline-7-carboxylic Acid

¹H NMR (300 MHz, [D₆]DMSO):

δ 0.35 (m, 1H, OH), 3.70 (m, 1H, 4′-Hₐ), 3.85-4.00 (m, 6H, 2′-H, 3′-H, 4′-H_{b}, 3 OH), 5.33 and 5.35 (2 s, together 1H, 1′-H of the isomers A and B), 7.98 and 8.01 (2 s, 1H, 5-H in isomer B, 8-H in isomer A), 8.30 and 8.33 (2 d, 1H, 6-H in isomer B, 7H in isomer A), 8.61 (s, 1H, 5-H in A, 8-H in B), 9.10 and 9.13 (2 s, 1H, 3-H of the isomers A and B).

¹³C NMR (75.5 MHz, [D₆]DMSO):

δ 64.9 (C-4′), 72.9 (C-3′), 73.8 (C-2′), 75.5 (C-1′), 129.2, 129.4 (C-5 in B, C-8 in A), 131.1 (C-5 in A, C-8 in B), 131.8, 132.3 (C-6 in B, C-7 in A 139.8, 142.0, 142.2, 143.6 (C-4a, C-8a), 146.6 (C-3), 160.3 (C-2),173.3 (COOH).

2-[(1′R,2′S,3′R)-4′-(α-D-Galactopyranosyloxy)-1′,2′,3′-trihydroxybutyl]-quinoxaline

¹H NMR (300 MHz, CD₃OD):

δ 3.31-3.86 (m, 7H, 3′-H, 2″-H, 3″-H, 4″-H, 5″-H, 6″-H₂), 4.01-4.06 (m, 3H, 2′-H, 4′-H₂), 4.88 (d, 1H, 1″-H), 5.35 (s, 1H, 1′-H), 7.76-7.83 (m, 2H, 6-H, 7-H), 8.04-8.08 (m, 2H, 5-H, 8-H), 9.14 (s, 1H, 3-H).—J_{1″,2″}=3.5 Hz.

¹³C NMR (75.5 MHz, CD₃OD): δ 62.7 (C-6″), 70.2 (C-4′), 70.5 (C-3′), 71.0 (C-4″), 71.6 (C-1′), 72.2 (C-3″), 73.7 (C-5″), 75.2 (C-2′),100.5 (C-1″), 129.6, 129.8 (C-5, C-8), 130.8, 131.4 (C-6, C-7), 142.5, 142.7 (C-4a, C-8a), 145.9 (C-3),159.7 (C-2).

MS (FD): m/e=435 [M+Na⁺].

2-[(1′R,2′S,3′R)-2′-(α-D-Glucopyranosyloxy)-1′,3′,4′-trihydroxybutyl]-quinoxaline

¹H NMR (300 MHz, CD₃OD):

δ 2.70-2.75 (m, 2H, 5″-H, 6″-Hₐ), 2.93-2.96 (m, 2H, 4″-H, 6″-H_{b}), 3.07 (dd, 1H, 2″-H), 3.19 (dd, 1H, 3″-H), 3.85 (d, 2H, 4′-H₂), 4.23 (d, 1H, 1″-H), 4.28 (dd, 1H, 2′-H), 5.35 (d, 1H, 1′-H), 7.78-7.85 (m, 2H, 6-H, 7-H), 8.05-8.09 (m, 2H, 5-H, 8-H), 9.11 (s, 1H, 3-H).—J_{1′,2′}=2.6, J_{2′,3′}=7.7, J_{3′,4′}=4.2, J_{1″,2″}=7.6, J_{2″,3″}=9.3 Hz.

¹³C NMR (75.5 MHz, CD₃OD):

δ 62.2 (C-6″), 63.9 (C-4′), 71.4 (C-4″), 72.5 (C-3′), 74.2 (C-1′), 77.1 (C-5″), 77.6 (C-3″), 82.5 (C-2′), 104.2 (C-1″), 129.5, 129.8 (C-6, C-7), 130.9, 131.3 (C-5, C-8),142.4 (C-4a, C-8a), 146.7 (C-3), 159.0 (C-2).

MS (FD): m/e=435 [M+Na⁺].

2-[(1′R,2′S,3′R)-2′-(α-D-Galactopyranosyloxy)-1′,3′,4′-trihydroxybutyl]-quinoxaline

¹H NMR (300 MHz, CD₃OD):

δ 2.44 (dd, 1H, 6″-Hₐ), 2.84 (dd, 1H, ⁶″-H_{b}), 3.00 (t, 1H, 5″-H), 3.32 (dd, 1H, 3″-H), 3.43 (dd, 1H, 2″-H), 3.61 (d, 1H, 4″-H), 3.86 (d, 2H, 4′-H₂), 3.97 (m, 1H, 3′-H), 4.19 (d, 1H, 1″-H), 4.23 (dd, 1H, 2′-H), 5.34 (d, 1H, 1′-H), 7.77-7.84 (m, 2H, 6-H, 7-H), 8.04-8.09 (m, 2H, 5-H, 8-H), 9.09 (s, 1H, 3-H).—J_{1′,2′}=2.6, J_{2′,3′}=7.7, J_{3′,4′}=4.0, J_{1″,2″}=7.7, J_{2″,3″}=9.6, J_{3″,4″}=3.0, J_{5″,6″Hb}=7.5, J_{5″,6″Ha}=5.7, J_{gem, 6″-H}_{₂}_{.}=10.8 Hz.

¹³C NMR (75.5 MHz, CD₃OD):

δ 58.7 (C-6″), 61.9 (C-4′), 67.3 (C-4″), 70.7 (C-3′), 70.8 (C-2″), 70.9 (C-1′), 72.8 (C-3″), 73.7 (C-5″), 81.2 (C-2′),103.1 (C-1″), 127.6, 127.8 (C-6, C-7), 128.9, 129.4 (C-5, C-8), 140.5, 140.6 (C-4a, C-8a), 144.6 (C-3), 157.1 (C-2).

MS (FD): m/e=413 [M+H⁺]; 435 [M+Na⁺].

### Example O

3-[4-(Methyloxycarbonyl)benzoyl]quinoxalin-2-one

Sodium p-toluenesulfinate (178 mg, 1.00 mmol), 1,3-dimethylimidazolium iodide (224 mg, 1.00 mmol), methyl 4-formylbenzoate (591 mg, 3.6 mmol) and sodium hydride (87 mg, 3.6 mmol) are added under an inert-gas atmosphere to a solution of dichloroquinoxaline (600 mg, 3.00 mmol) in 20 ml of DMF (dimethylformamide), anhydrous, and the mixture is warmed to a temperature of 80° C. After 3 hours, the reaction mixture is poured onto ice, neutralised using glacial acetic acid and subsequently extracted with ethyl acetate. After the organic phase has been washed with water and dried over MgSO₄, the solvent is removed, and the residue is purified by column chromatography on silica gel using an eluent mixture (cyclohexane/ethyl acetate (1:1). The pale-yellow crystalline ketoquinoxaline is obtained in a yield of 57%.

Characterisation:

IR (KBr):

| | | |
|---|---|---|
| Wavenumber | 3065 | (ν(C—H), Ph) |
| [cm − 1] = | | |
| | 2967 | (ν(C—H), CH₃) |
| | 1736 | (ν(C—H), CO₂Me) |
| | 1696 | (ν(C—H), PhCO) |
| | 1624 | (ν(C—H), Ph) |
| | 1289 | ((skeleton), Ph) |
| | 814 | (γ(C—H), Ph) |
| | 735 | (γ(C—H), Ph) |

300 MHz-¹H—NMR (CDCl₃):

| | | |
|---|---|---|
| δ = | 11.91 | (br. s, 1H, N—H) |
| | 8.08-8.14 | (m, 4H, hetaryl arom.) |
| | 7.07-7.15 | (m, 4H, arom.) |
| | 3.89 | (s, 3H, CH₃) |

75 MHz-¹³C—NMR (CDCl₃):

| | | |
|---|---|---|
| δ = | 207.48 | ((C═O) PhCO) |
| | 166.49 | ((C═O) C-2) |
| | 165.54 | ((Cquat) C-3) |
| | 155.11 | ((CO) CO₂He) |
| | 137.63 | (C_{quat} arom.) |
| | 134.72 | (C_{quat} arom.) |
| | 133.08 | (C_{quat} arom.) |
| | 129.54 | (C—H arom.) |
| | 129.29 | (C—H arom.) |
| | 128.01 | (C—H arom.) |
| | 125.52 | (C—H arom.) |
| | 125.42 | (C—H arom.) |
| | 122.93 | (C—H arom.) |
| | 115.04 | (C—H arom.) |
| | 52.42 | (CH₃) |

| | |
|---|---|
| MS(70eV): | |
| m/e (%) = | 308 [M+] (11), 249 (5), 172 (4), 162 (100), 149 (1), 134 |
| | (80), 120 (1), 106 (66), 91 (19), 79 (45) |

### Example P

The following table shows the structural formulae of quinoxaline derivatives which can be employed in accordance with the invention and the maxima of their UV-A and UV-B absorption. The measurement was carried out in 2-propanol at a concentration of 1 mg of substance per 100 ml of solvent.

| | UV-A (400 320 nm) | | UV-B (280 320 nm) | |
|---|---|---|---|---|
| Structural formula | Max. abs. | λ [nm] | Max. abs. | λ [nm] |
| 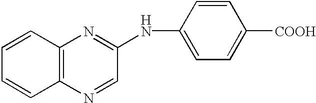 | 0.378 | 374.1 | 1.141 | 297.2 |
| 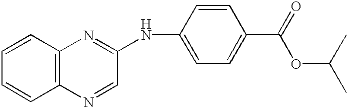 | 0.555 | 374.3 | 1.262 | 306.7 |
| 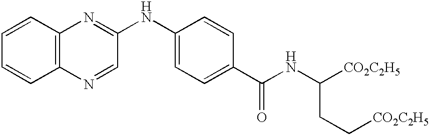 | 0.3076 | 380.0 | 0.756 | 307.0 |
| 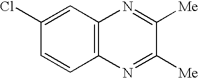 | 0.400 | 322.0 | | |
| 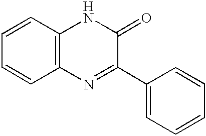 | 0.360 | 365.0 | 0.373 | 305.0 |
| 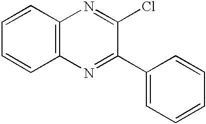 | 0.173 | 335.0 | | |
| 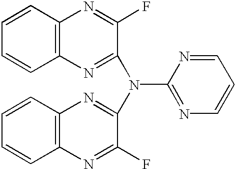 | 0.446 | 329.8 | | |
| 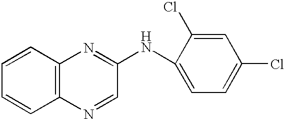 | 0.248 | 365.2 | 0.757 | 280.7 |
| 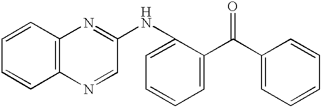 | 0.424 | 395.4 | — | — |
| 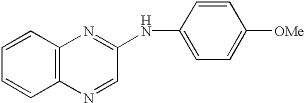 | 0.306 | 386.9− | 0.837 | 291.1 |
| 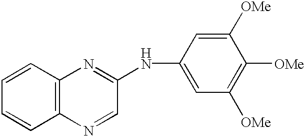 | 0.281 | 386.1 | 0.679 | 275.6 |
| 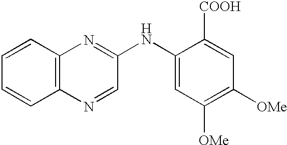 | 0.456 | 380.1 | 0.455 | 300.1 |
| 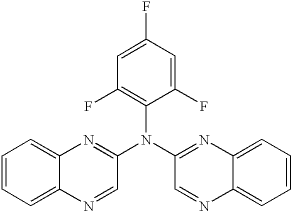 | | | 0.966 | 289.3 |
| 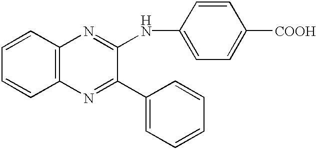 | 0.325 | 376.8 | 0.452 | 303.8 |
| 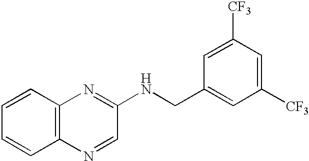 | 0.192 | 359.0 | | |
| 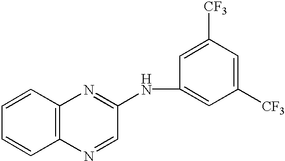 | 0.262 | 359.8 | | |
| 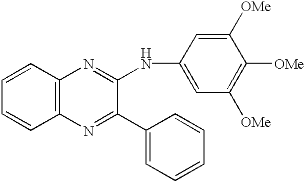 | 0.175 | 390.4 | 0.475 | 287.6 |
| 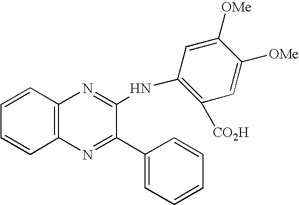 | 0.235 | 393.4 | 0.635 | 285.6 |
| 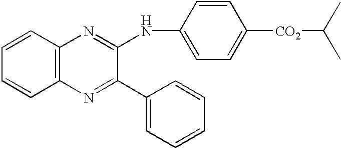 | 0.342 | 381.9 | 0.849 | 305.3 |
| 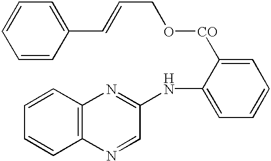 | 0.469 | 386.3 | | |
| 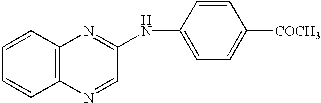 | 0.622 | 374.9 | 1.062 | 314.7 |
| 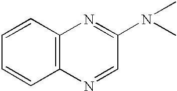 | 0.413 | 378.0 | | |
| 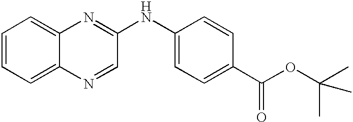 | 0.506 | 374.3 | 1.172 | 306.3 |
| 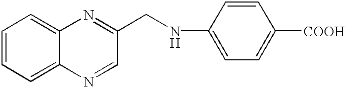 | | | 0.837 | 302.9 |
| 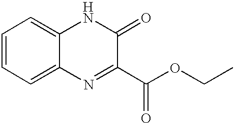 | 0.245 | 355.8 | 0.279 | 290.0 |
| 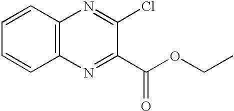 | 0.243 | 322.9 | | |
| 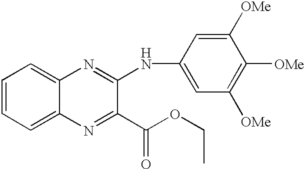 | | | 0.614 | 295.6 |
| 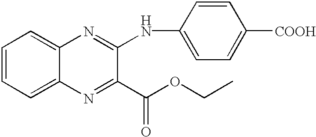 | | | 1.270 | 311.5 |
| 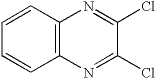 | 0.3379 | 336.5 | | |
| 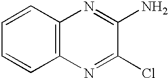 | 0.427 | 353.4 | | |
| 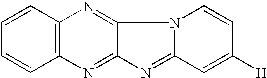 | 0.411 | 348.3 | | |
| 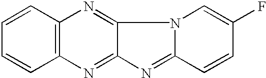 | 0.132 | 344.6 | | |
| 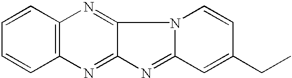 | 0.536 | 356.1 | | |
| 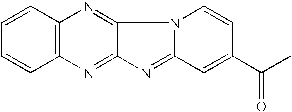 | 0.533 | 364.8 | | |
| 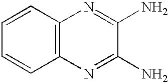 | 0.6269 | 342.5 | | |
| 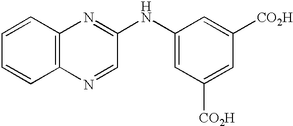 | 0.319 | 372.8 | 0.887 | 286.9 |
| 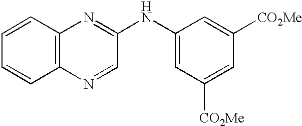 | 0.322 | 368.3 | 0.789 | 287.7 |
| 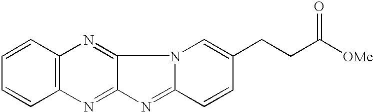 | 0.3747 | 349.5 | | |
| 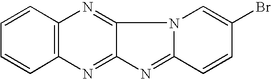 | 0.4113 | 354 | | |
| 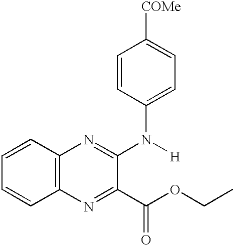 | 1.2265 | 328.5 | | |
| 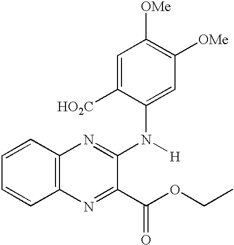 | | | 0.5713 | 295.0 |
| 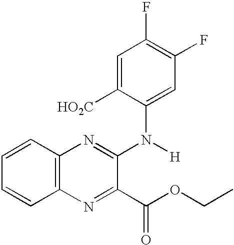 | 0.1187 | 381.5 | 0.4067 | 287.0 |
| 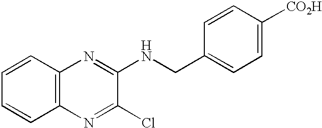 | 0.2348 0.2330 | 333.0 346.0 | 0.2183 | 290.0 |
| 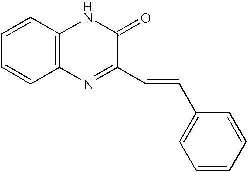 | 0.7324 | 388.5 | | |
| 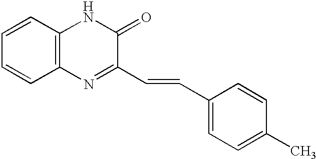 | 0.4660 | 336.0 | | |
| 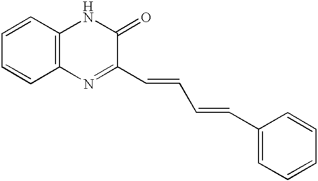 | | | 0.6004 | 281.5 |
| 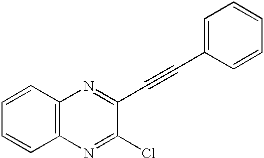 | 0.5539 0.5439 | 366.0 350.5 | 0.6431 | 286.0 |
| 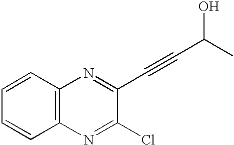 | 0.4653 0.4557 | 352.0 338.5 | | |
| 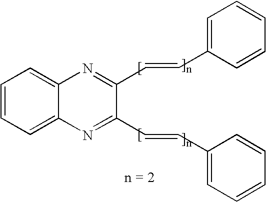 | 0.8610 0.8531 | 363.5 327.5 | | |
| 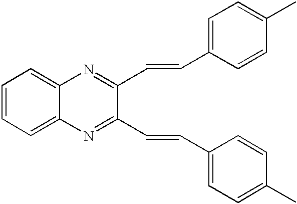 | | | 0.3327 | 316.0 |
| 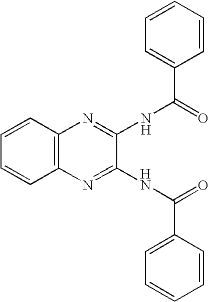 | 0.7643 0.7984 | 368.0 352.0 | | |
| 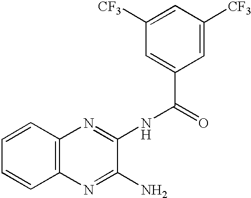 | 0.4130 | 354.0 | | |
| 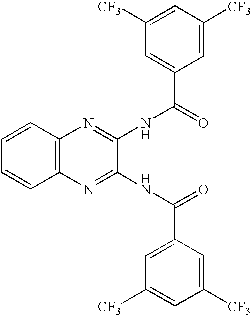 | 0.1734 0.1826 | 349.0 340.0 | | |
| 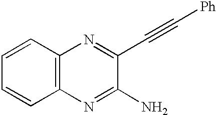 | 0.5137 0.5043 | 395.3 321.0 | 0.3618 0.3968 | 257.0 202.0 |
| 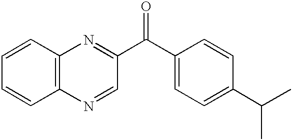 | — | — | — | — |
| 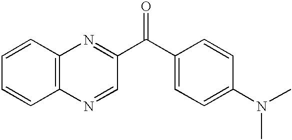 | 0.3405 0.4828 | 387.0 322.0 | | |
| 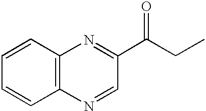 | | | 0.3236 | 307.0 |
| 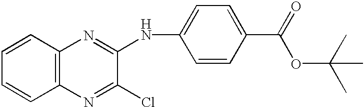 | 0.2677 0.3342 0.2688 0.5593 0.5519 | 391.0 373.0 356.0 335.0 325.0 | 0.4672 | 291.0 |
| 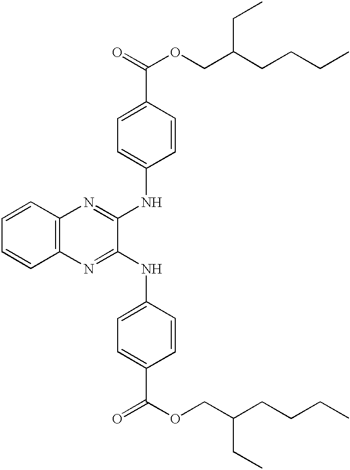 | 0.4647 0.4269 | 336.0 323.0 | | |
| 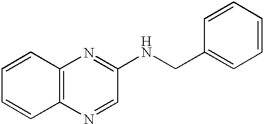 | 0.2602 | 364.0 | | |
| 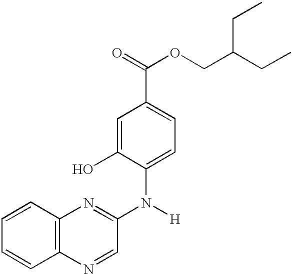 | 0.4059 0.5296 | 381.0 320.0 | 0.5569 | 291.0 |
| 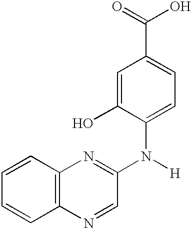 | 0.5594 | 381.0 | 0.7514 0.8969 | 319.0 290.0 |
| 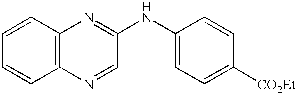 | 0.5414 | 375.0 | 1.2839 | 306.0 |
| 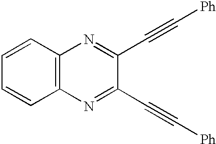 | 0.4443 | 385.0 | 0.8366 | 294.0 |
| 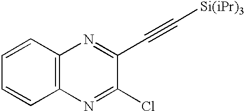 | 0.4936 0.4868 | 355.0 340.0 | | |
| 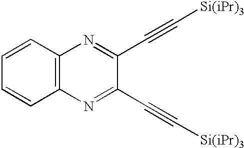 | 0.2594 0.2348 | 375.0 358.0 | 1.0488 | 272.0 |
| 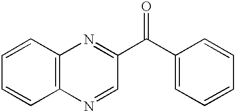 | | | 0.330 | 300.0 |
| 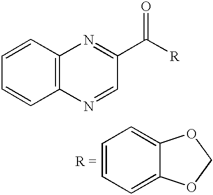 | | | 0.3349 | 336.0 |
| 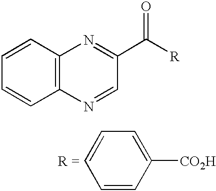 | 0.4206 | 307.0 | | |
| 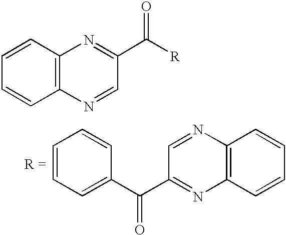 | | | 0.2798 | 317.0 |
| 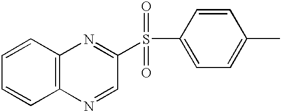 | 0.1595 | 321.0 | | |
| 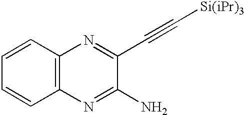 | 0.1707 | 390.0 | 0.1782 | 311.0 |
| 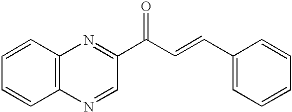 | 0.1731 | 329.0 | | |
| 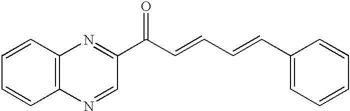 | 1.0497 | 365.0 | 0.6584 | 302.0 |
| 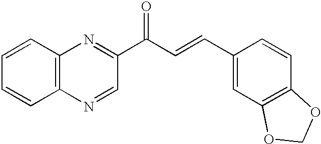 | 0.5101 | 381.0 | 0.4556 | 305.0 |
| 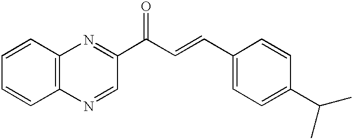 | 0.7731 | 343.0 | | |
| 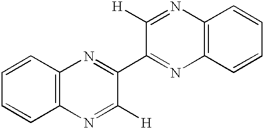 | 0.7687 | 341.0 | | |
| 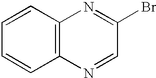 | 0.4424 | 321.0 | | |
| 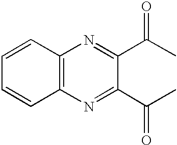 | | | 0.3011 | 310.0 |
| 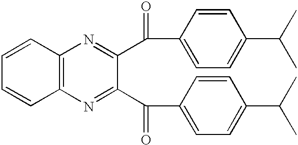 | | | 0.45 | 280.0 |
| 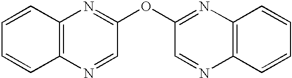 | 0.2587 | 320.0 | | |
| 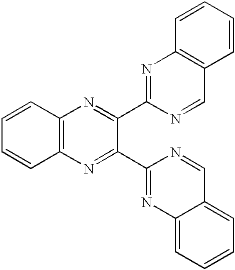 | 0.5570 0.4203 | 335.0 333.0 | | |

### USE EXAMPLES

### Example 1

Preparation of a sunscreen spray according to the invention (O/W)

| | | % |
|---|---|---|
| A | | |
| Isopropyl N-(2-quinoxalinyl)-4-aminobenzoate | (1) | 1.00 |
| Eusolex 2292 (Art. No. 105382) | (1) | 7.50 |
| (Octyl Methoxycinnamate) | | |
| Eusolex HMS (Art. No. 111412) | (1) | 7.00 |
| (Homosalate) | | |
| Volpo S-2 (Steareth-2) | (2) | 0.40 |
| Volpo S-10 (Steareth-10) | (2) | 0.80 |
| Pemulen TR-2 | (3) | 0.18 |
| (Acrylate/C10-39Alkyl Acrylate Crosspolymer) | | |
| Hetester PHA | (4) | 5.00 |
| (Propylene Glycol Isoceteth-3 Acetate) | | |
| Performa V 825 | (5) | 0.80 |
| (Synthetic Wax) | | |
| Dow Corning 200 (100cs) | (6) | 1.00 |
| (Dimethicone) | | |
| Oxynex K liquid (Art. No. 108324) | (1) | 0.10 |
| (PEG-8 (and) Tocopherol (and) Ascorbyl Palmitate (and) | | |
| Ascorbic Acid (and) Citric Acid) | | |
| B | | |
| Eusolex 232 (Art. No. 105372) | (1) | 1.00 |
| (Phenylbenzimidazole Sulfonic Acid) | | |
| Triethanolamine (Art. No. 108379) | (1) | 0.90 |
| Propane-1,2-diol (Art. No. 107478) | (1) | 2.00 |
| Preservatives | | q.s. |
| Water, demineralised | ad | 100.00 |

Preparation

For the neutralisation of Eusolex 232, the triethanolamine is added to the water of phase B, and Eusolex 232 is added with stirring. After complete dissolution, the remaining raw materials of phase B are added and heated to 80° C. Combine phase A apart from the Pemulen and heat to 80° C. Stir Pemulen into phase A. Add phase B slowly to phase A with stirring, homogenise and cool with stirring.

Notes

The following preservatives are used:

0.05% of propyl 4-hydroxybenzoate (Art. No. 107427)

0.15% of methyl 4-hydroxybenzoate (Art. No. 106757)

Sources of Supply

(1) Merck KGaA, Darmstadt

(2) Croda, Nettetal

(3) Goodrich, Neuss

(4) ROVI, Schlüchtern

(5) New Phase, N.J. 08554

(6) Dow Corning, Wiesbaden

### Example 2

Preparation of a sunscreen gel according to the invention (O/W)

| | | % |
|---|---|---|
| A | | |
| Isopropyl N-(2-quinoxalinyl)-4-aminobenzoate | (1) | 1.00 |
| Eusolex 6300 (Art. No. 5385) | (1) | 10.00 |
| Luvitol EHO | (2) | 2.00 |
| Dow Corning 200 (100 cs) | (3) | 2.00 |
| Shea butter | (4) | 5.00 |
| Antaron V-220 | (5) | 2.00 |
| Oxynex K liquid (Art. No. 8324) | (6) | 1.00 |
| B | | |
| Eusolex 232 (Art. No. 5372) | (1) | 0.75 |
| Tris(hydroxymethyl)aminomethane (Art. No. 8386) | (1) | 0.33 |
| Preservatives | | q.s. |
| Water, demineralised | | 20.00 |
| C | | |
| Tris(hydroxymethyl)aminomethane (Art. No. 8386) | (1) | 1.20 |
| Water, dem. | | 10.00 |
| D | | |
| Pemulen TR-1 | (6) | 0.60 |
| Water, dem. | ad | 100.00 |

Preparation

For phase D, disperse the Pemulen TR-1 homogeneously in the water and add the pre-dissolved phase C with stirring. For the neutralisation of Eusolex 232, the tris(hydroxymethyl)aminomethane is dissolved in the water of phase B, and the Eusolex 232 is added with stirring. After complete dissolution, the remaining raw materials of phase B are added. Stir phase B into phase C/D. Dissolve phase A with warming and stir into phase B/C/D. Homogenise.

Notes

The following preservatives are employed:

0.05% of propyl 4-hydroxybenzoate (Merck Art. No. 7427)

0.15% of methyl 4-hydroxybenzoate (Merck Art. No. 6757)

Sources of Supply

(1) Merck KGaA, Darmstadt

(2) BASF, Ludwigshafen

(3) Dow Corning, Düsseldorf

(4) H. Erhard Wagner, Bremen

(5) GAF, Frechen

(6) Goodrich, Neuss

### Example 3

Preparation of a sunscreen lotion (W/O) with UV-A/B protection

| | | % |
|---|---|---|
| A | | |
| Isopropyl N-(2-quinoxalinyl)-4-aminobenzoate | (1) | 1.00 |
| Eusolex 2292 (Art. No. 1.05382) | (1) | 3.00 |
| Eusolex 4360 (Art. No. 1.05376) | (1) | 2.00 |
| (Benzophenone-3) | | |
| Dehymuls E | (2) | 6.00 |
| (Dicocoyl Pentyerythrityl Citrate (and) Sorbitan | | |
| Sesquioleate (and) Cera Alba (and) Aluminium Stearate) | | |
| Arlacel 989 | (3) | 1.00 |
| (PEG-7 Hydrogenated Castor Oil) | | |
| Beeswax (Art. No. 1.11544) | (2) | 2.00 |
| Cetiol J 600 | (2) | 6.00 |
| (Oleyl Erucate) | | |
| Cetiol V | (2) | 6.00 |
| (Decyl Oleate) | | |
| Cetiol OE | (2) | 5.00 |
| (Dicaprylyl Ether) | | |
| Dow Corning 200 (100 cs) | (4) | 1.00 |
| (Dimethicone) | | |
| B | | |
| Glycerol (about 87%) (Art. No. 1.04091) | (1) | 5.00 |
| Magnesium sulfate heptahydrate (Art. No. 1.05882) | (1) | 1.00 |
| Preservatives | | q.s. |
| Water, dem. | ad | 100.00 |

Preparation

Heat phase B to 80° C. and phase A to 75° C. Stir phase B slowly into phase A. Homogenise and cool with stirring.

Notes

The following preservatives are included:

0.05% of propyl 4-hydroxybenzoate (Art. No. 1.07427)

0.15% of methyl 4-hydroxybenzoate (Art. No. 1.06757)

Sources of Supply

(1) Merck KGaA, Darmstadt

(2) Henkel KGaA, Düsseldorf

(3) ICI, Essen

(4) Dow Corning, Düsseldorf

### Example 4

Preparation of a sunscreen lotion containing IR3535™

| | | % |
|---|---|---|
| A | | |
| N-(2-Quinoxalinyl)-4-aminobenzoic acid | (1) | 3.00 |
| Eusolex 6300 | (1) | 1.00 |
| (4-Methylbenzyliden Camphor) | | |
| IR 3535 ™ (Art. No. 111887) | (1) | 10.00 |
| (Ethyl Butylacetylaminopropionate) | | |
| (-)-α-Bisabolol (Art. No. 130170) | (1) | 0.30 |
| Montanov 68 | (2) | 4.00 |
| (Cetearyl Alcohol (and) Cetearyl Glucoside) | | |
| Myritol 312 | (3) | 2.00 |
| (Carprylic/apric Triglyceride) | | |
| Mirasil CM 5 | (4) | 2.00 |
| (Cyclomethicone) | | |
| Mirasil DM 350 | (4) | 1.00 |
| (Dimethicone) | | |
| B | | |
| Demin. water | ad | 100.00 |
| Glycerol, 87% (Art. No. 104091) | (1) | 3.00 |
| Preservatives | | q.s. |
| C | | |
| Rhodicare S | (4) | 0.50 |
| (Xanthan Gum) | | |

Preparation

Heat phases A and B separately to 75° C. Add C slowly to B at 75° C. with stirring and stir until a homogeneous mixture has formed. Subsequently add A to B/C with stirring and homogenise. Cool with stirring.

Notes

The following preservatives are included:

0.05% of propyl 4-hydroxybenzoate (Merck KGaA, Art. No. 130173),

0.15% of methyl 4-hydroxybenzoate (Merck KGaA, Art. No. 130174),

0.30% of Germall 115 (ISP, Frechen)

Sources of Supply

(1) Merck KGaA, Darmstadt

(2) Interorgana, Cologne

(3) Henkel, KGaA, Düsseldorf

(4) Rhodia, Frankfurt

### Example 5

Preparation of a sunscreen milk containing IR3535™

| | | % |
|---|---|---|
| A | | |
| N-(2-Quinoxalinyl)-4-aminobenzoic acid | (1) | 1.00 |
| Eusolex 6300 | (1) | 1.00 |
| (4-Methylbenzyliden Camphor) | | |
| IR 3535 ™ (Art. No. 111887) | (1) | 15.00 |
| (Ethyl Butylacetylaminopropionate) | | |
| Eusolex 2292 | (1) | 3.00 |
| (Octyl Methoxycinnamate) | | |
| Dow Corning 5225 C | (2) | 12.00 |
| (Cyclomethicone (and) Dimethicone Copolyol) | | |
| Dow Corning 345 | (2) | 5.00 |
| (Cyclomethicone) | | |
| Gilugel Sil 5 | (3) | 12.00 |
| (Cyclomethicone (and) Al/Mg Hydroxyde Stearate) | | |
| Solvent ID | (4) | 13.00 |
| (Isododecane) | | |
| Wiconol 14 | (5) | 2.50 |
| (Polyglyeryl-4 Oleate) | | |
| Beeswax, bleached (Art. No. 111544) | (1) | 1.60 |
| Carnauba wax | (6) | 0.40 |
| B | | |
| Demin. water | ad | 100.00 |
| Propane-1,2-diol (Art. No. 107478) | (1) | 2.00 |
| Sodium chloride (Art. No. 106400) | (1) | 2.00 |
| Preservatives | (1) | q.s. |
| C | | |
| Bariton perfume oil (10607) | (7) | 0.30 |

Preparation

Heat phases A to 80° C., cool to 30° C. with stirring. Add phase B slowly to phase A with stirring, stir until a homogeneous mixture has formed and homogenise. Add phase C.

Notes

The following preservative is included:

0.20% of Euxyl K400 (Schülke & Mayr, Norderstedt)

Sources of Supply

(1) Merck KGaA, Darmstadt

(2) Dow Corning, Düsseldorf

(3) Nordmann, Rassmann GmbH&Co, Hamburg

(4) BP, Düsseldorf

(5) Witco Chemical, Frankfurt

(6) Aug. Schmidt Nachfolger, Bremen

(7) Haarmann & Reimer, Holzminden

1. Use of quinoxaline derivatives of the formulae I, II and/or III 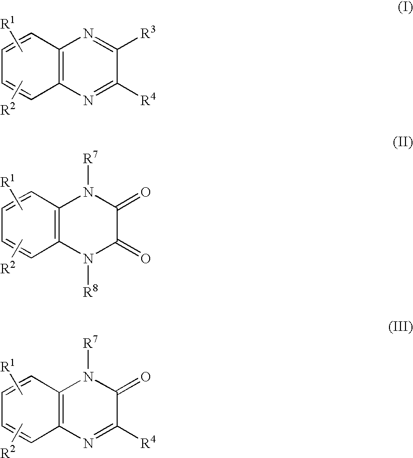
in which
R¹and R² are each, independently of one another, H,
alkyl, alkoxy, alkenyl or alkynyl, each having up to 20 carbon atoms, cycloalkyl, cycloalkoxy, cycloalkenyl or bicyclic systems, each having up to 10 carbon atoms, where, in each of these groups, one or more hydrogen atoms may also be substituted by Sub¹ and/or one or two CH₂ groups may be replaced by C═O, and the cyclic systems may contain from 1 to 3 heteroatoms, such as S, N and/or O,
Hal, OH, NO₂, —(CR⁵R⁶)ₙ—NR⁵R⁶, —(CR⁵R⁶)ₙ—N═CR⁵R⁶, —(CR⁵R⁶)ₙ—CR⁵═NR⁵, —(CR⁵R⁶)ₙ—NHCOR⁵, —(CR⁵R⁶)ₙ—NHCOOR⁵, —SR⁵, —SO₂—R⁵ or NR⁵—SO—R⁶, —SO—R⁵,
water-solubilising substituents selected from the group consisting of carboxylate, sulfonate or ammonium radicals,
COR⁵, COOR⁵, COR⁵R⁶, CN, O═S(—R⁵)═O O═S(—OR⁵)═O, O═S(—NR⁵R⁶)═O, R⁵OP(—OR⁶)═O, OAr, —(CR⁵R⁶)ₙ—Ar,
-Het, —NHHet, —OHet or —(CR5R6)ₙ-Het,
R¹and R² together, also with carbon atoms to which they are bonded, may jointly form an unsaturated, partially saturated or fully saturated 4-, 5-, 6- or 7-membered ring, which may optionally contain heteroatoms, such as S, N and/or O, may be further fused and/or may also be monosubstituted or polysubstituted,
Sub¹ is Hal, hydroxyl, cyano, amino, nitro, C₁-C₄-alkylamino, C₁-C₄-dialkylamino, C₁-C₄-alkyl or C₁-C₄-alkoxy, COOH or COO-alkyl,
Hal is fluorine, chlorine, bromine or iodine,
n is 0, 1, 2, 3 or 4,
R⁵ and R⁶ are each, independently of one another, H,
alkyl, alkenyl or alkynyl, each having up to 20 carbon atoms, cycloalkyl, cycloalkenyl or bicyclic systems, each having up to 10 carbon atoms, where these radicals may be up to trisubstituted by Sub¹, and/or one or two CH₂ groups may be replaced by C═O, and the cyclic systems may also contain from 1 to 3 heteroatoms, such as S, N and/or O,
—(CR^{′}R^{″})ₙ—Ar or —(CR^{′}R^{″})ₙ-Het,
the radicals R⁵ and R⁶ may also jointly form with one another, in each case together with carbon atoms to which they are bonded, an unsaturated, partially saturated or fully saturated 4-, 5-, 6- or 7-membered ring, which may optionally contain heteroatoms, such as S, N and/or O, may also be monosubstituted or polysubstituted and/or may be further fused,
R^{′} and R^{″} are each, independently of one another, H or C₁-C₄-alkyl, where one or two CH₂ groups may also be replaced by C═O,
Ar is an unsubstituted or monosubstituted or polysubstituted aromatic ring or fused ring systems having from 6 to 18 carbon atoms, in which, in addition, one or two CH groups may be replaced by C═O,
Het is an unsubstituted or monosubstituted or polysubstituted heteroaromatic ring having from 5 to 7 ring members or a fused ring system, in which one or more N, S and/or O atoms are present as heteroatoms and in which, in addition, one or two CH groups in the α- or β-position to the heteroatoms may be replaced by C═O,
R³ and R⁴ are each, independently of one another, H,
alkyl, alkoxy, alkenyl or alkynyl, each having up to 20 carbon atoms, cycloalkyl, cycloalkoxy, cycloalkenyl or bicyclic systems, each having up to 10 carbon atoms, where these radicals may be up to trisubstituted by Sub² and/or one or two CH₂ groups may be replaced by C═O and where the cyclic systems may also contain from 1 to 3 heteroatoms, such as S, N and/or O,
Hal, OH, NO₂, —(CR⁵R⁶)ₙ—NR⁵R⁶, —(CR⁵R⁶)ₙ—N═CR⁵R⁶, —(CR⁵R⁶)ₙ—CR⁵═NR⁵, —(CR⁵R⁶)ₙ—NHCOR⁵, —(CR⁵R⁶)ₙ—NHCOOR⁵, —SR⁵, —SO₂—R⁵, NR⁵—SO—R⁶ or —SO—R⁵,
water-solubilising substituents selected from the group consisting of carboxylate, sulfonate or ammonium radicals,
COR⁵, COOR⁵, CONR⁵R⁶, CN, O═S(—R⁵)═O, O═S(—OR⁵)═O, O═S(—NR⁵R⁶)═O, R⁷OP(—OR⁸)═O, OAr, —(CR⁵R⁶)ₙ—Ar,
-Het, —NHHet, —OHet or —(CR⁵R⁶)ₙ-Het,
the radicals R³ and R⁴ may also jointly form with one another, in each case together with the carbon atoms to which they are bonded, or alternatively one of the two radicals together with the adjacent N atom, an unsaturated, partially saturated or fully saturated 4-, 5-, 6- or 7-membered ring, which may optionally contain heteroatoms, such as S, N and/or O, may also be monosubstituted or polysubstituted and/or may be further fused,
R⁷ and R⁸ are each, independently of one another, H,
alkyl, alkoxy, alkenyl or alkynyl, each having up to 20 carbon atoms, cycloalkyl, cycloalkoxy, cycloalkenyl or bicyclic systems, each having up to 10 carbon atoms, where these radicals may be up to trisubstituted by Sub² and/or one or two CH₂ groups may be replaced by C═O and where the cyclic systems may also contain from 1 to 3 heteroatoms, such as S, N and/or O,
Sub² is Hal, hydroxyl, cyano, amino, nitro, C₁-C₄-alkyl or C₁-C₄-alkoxy, COR⁵, COOR⁵, OAr, OHet, —(CR⁵R⁶)ₙ—Ar or —(CR⁵R⁶)ₙ-Het, —(CR⁵R⁶)ₙ—NR⁵R⁶, CONR⁵R⁶, CN, O═S(—R⁵)═O, O═S(—OR⁵)═O, O═S(—NR⁵R⁶)═O or R⁷OP(—OR⁸)═O,
as photostable UV filters in cosmetic and pharmaceutical preparations for protection of the human skin or human hair against solar radiation, alone or together with UV-absorbent compounds known per se for cosmetic and pharmaceutical preparations.

2. Use of quinoxaline derivatives of the formulae Ii and/or Ij according to the formula I according to claim 1, 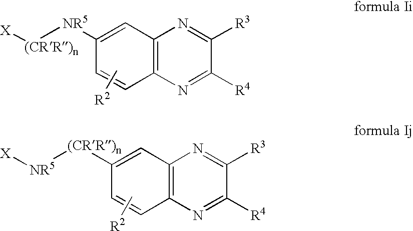
in which R², R³, R⁴, R⁵, R′, R″ and n are as defined in claim 1, and X is Ar or Het with the definitions given therefor in claim 1.

3. Use of quinoxaline derivatives of the formulae Ik and/or Il according to the formula I according to claim 1, 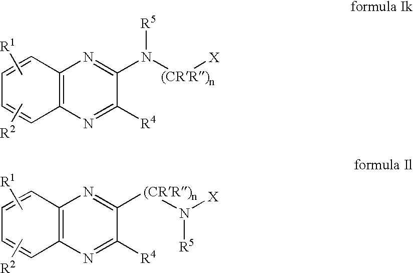
in which R², R¹, R⁴, R⁵, R′, R″ and n are as defined in claim 1, and X is Ar or Het with the definitions given therefor in claim 1.

4. Compound of the formula Ik or Il 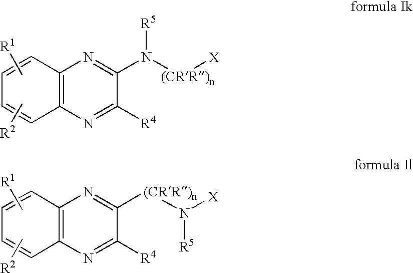
in which R², R¹, R⁴, R⁵, R′, R″ and n are as defined in claim 1, and X is Ar or Het with the definitions given therefor in claim 1.

5. Compounds from the group consisting of:
5-nitro-2,3,6-trimethoxyquinoxaline;
2,3,7-trimethoxy-6,8-dinitroquinoxaline;
isopropyl N-(2-quinoxalinyl)-4-aminobenzoate;
N,N-benzoyl-(2-quinoxalinyl)-4-aminobenzoic acid;
N-(2-quinoxalinyl)4-aminomethylbenzoic acid;
N-(2-quinoxalinyl)-2-amino-4,5-dimethoxybenzoic acid;
N-(2-quinoxalinyl)-2-aminopyrimidine;
N-(2-quinoxalinyl)-2-aminobenzophenone;
N-(2-quinoxalinyl)-4-aminoanisole;
N-(2-quinoxalinyl)-3,4,5-trimethoxyaniline;
N-[bis(2-quinoxalinyl)]-2,4,6-trifluoroaniline;
N-[2-(3-phenyl)quinoxalinyl]-4-aminobenzoic acid.

6. Use of compounds of the formulae I, II and/or III as UV stabilisers in cosmetic and pharmaceutical preparations.

7. Light-protection composition comprising cosmetic and pharmaceutical preparations for protection of the human epidermis or human hair against UV light in the range from 280 to 400 nm, characterised in that they comprise, in a cosmetically and pharmaceutically suitable carrier, alone or together with UV-absorbent compounds known per se for cosmetic and pharmaceutical preparations, effective amounts of compounds of the formulae I, II and/or III 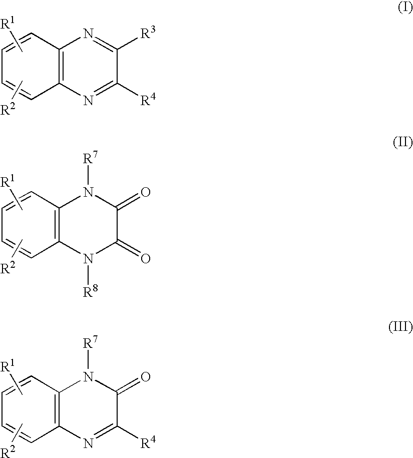
in which the, variables are as defined in claim 1, as photostable UV filters.

8. Light-protection compositions comprising cosmetic and pharmaceutical preparations according to claim 7, comprising compounds of the formulae I, II and/or III, in which the variables are as defined in claim 2 and/or 3, as UV filters.

9. Cosmetic or pharmaceutical preparation according to at least one of the preceding claims, where the preparation comprises one or more UV filters, which are preferably selected from the group consisting of 3-(4′-methylbenzylidene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)-benzoate, 2-ethylhexyl 2-cyano-3,3-diphenylacrylate, 2-phenylbenzimidazole-5-sulfonic acid and its potassium, sodium and triethanolamine salts.

10. Cosmetic or pharmaceutical preparation according to at least one of the preceding claims, characterised in that one or more antioxidants and/or at least one of the pyrimidinecarboxylic acids ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid) and/or at least one aryl oxime, preferably 2-hydroxy5-methyllaurophenone oxime, and/or a coumaranone derivative, preferably 4,6,3′,4′-tetrahydroxybenzyl-3-coumaranone or its trisulfate, are included.

11. Use of a compound of the formulae I, II and/or III according to claim 1 in combination with antioxidants in cosmetic or pharmaceutical preparations.
